(19) United States
(12) Patent Application Publication
(10) Pub. No.: US 2024/0310383 A1
(43) Pub. Date: Sep. 19, 2024

(54) Proteomic Risk Score For Osteoarthritis (OA)

(71) Applicant: Regeneron Pharmaceuticals, Inc., Tarrytown, NY (US)

(72) Inventors: Charles Paulding, Tarrytown, NY (US); Nan Lin, Tarrytown, NY (US); Lei Chen, Tarrytown, NY (US)

(21) Appl. No.: 18/606,302

(22) Filed: Mar. 15, 2024

Related U.S. Application Data
(60) Provisional application No. 63/452,729, filed on Mar. 17, 2023.

(51) Int. Cl. G01N 33/68

(52) U.S. Cl. CPC G01N 33/6887; G01N 2333/78; G01N 2800/105

(57) ABSTRACT

Methods of determining the risk of developing osteoarthritis (OA) and/or progression to total joint replacement (TJR) and determining the treatment response to an NGF antagonist or NSAID treatment in a subject by determination of an OA proteomic risk score for the subject are presented herein.

### FIELD

The present disclosure is directed to methods of determining the risk of developing osteoarthritis (OA) and/or progression to total joint replacement (TJR) and determining the treatment response to a Nerve Growth Factor (NGF) antagonist or a Non-Steroidal Anti-Inflammatory Drug (NSAID) in a subject by determination of an OA proteomic risk score for the subject.

### BACKGROUND

Proteomic risk scores combine information of protein expression levels or circulating levels from a number of proteins derived from disease association studies to create a single quantitative measure for each individual. A proteomic risk score is typically constructed as the weighted sum of the expression level or circulating level of a selected collection of proteins from subjects with a particular disease, compared from the reference population. The resulting score is approximately normally distributed in the general population, with higher scores indicating higher risk or disease and/or need for a particular therapeutic treatment regimen. With the increasing availability of proteomic data in large cohort studies, inclusion of proteomic risk scores may become more widespread. Previously this required specialist knowledge, but as tools and data availability have improved it has become more feasible to calculate scores for use in analyses.

Osteoarthritis is the most common form of arthritis, affecting an estimated 303 million people globally in 2017, and pain is the predominant symptom associated with this disease. OA is a degenerative disease of the synovial joints including the knee, hip, facet joints of the spine, and hand. Risk factors for OA include aging, prior joint injury, obesity, female sex, and genetics. These risk factors are related to the underlying pathogenesis of OA, which is a complex process impacted by altered biomechanics, chronic low-level inflammation, and aging. All of these processes can promote degradation and remodeling of the joint tissues, which ultimately results in failure of the structural integrity of the joint. Although these arthropathy and arthritis do not directly lead to death, they are painful due to the progress of cartilage and bone destruction over time and cause limb dysfunction, which has a great impact on daily life.

R475-OA-1688 is a phase 3 randomized, double-Blind, multi-dose, placebo and NSAIDs-controlled study to evaluate the efficacy and safety of fasinumab in patients with pain due to osteoarthritis of the knee or hip. A primary efficacy objective of R475-OA-1688 is to evaluate the change in Western Ontario and McMaster Osteoarthritis (WOMAC) pain subscale and functional subscale from baseline to week 24 in patients treated with fasinumab compared with patients treated with NSAIDs or placebo. A primary safety objective of R475-OA-1688 is to access the incidence of total joint replacement during treatment and follow-up periods.

### SUMMARY

The present disclosure provides methods of determining the risk of a subject for developing OA, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has an increased risk of developing OA; and when the subject's osteoarthritis proteomic risk score is less than a threshold osteoarthritis proteomic risk score, the subject has a decreased risk of developing OA.

The present disclosure also provides methods of determining the risk of a subject progressing to total joint replacement (TJR), the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has an increased risk of progressing to TJR; and when the subject's osteoarthritis proteomic risk score is less than a threshold osteoarthritis proteomic risk score, the subject has a decreased risk of progressing to TJR.

The present disclosure also provides methods of determining the treatment response of a subject treated with an NGF antagonist, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has less response to an NGF antagonist; and when the subject's osteoarthritis proteomic risk score is less than a threshold osteoarthritis proteomic risk score, the subject has an increased response to an NGF antagonist.

The present disclosure also provides methods of determining the treatment response of a subject treated with an NSAID, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has diminishing response to an NSAID; and when the subject's osteoarthritis proteomic risk score is less than a threshold osteoarthritis proteomic risk score, the subject has an increased response to an NSAID.

The present disclosure also provides methods of determining the treatment response of a subject, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has less treatment response; and when the subject's osteoarthritis proteomic risk score is less than a threshold osteoarthritis proteomic risk score, the subject has an increase in treatment response.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Total Joint Replacement percentage (Y axis) in OA patients from the UK Biobank Pharma Proteomics Project (UKBPPP), stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 2 shows Total Joint replacement Kaplan-Meier curves in UK Biobank Pharma Proteomics Project (UKB PPP) OA patients stratified by proteomic risk score quartiles; quartile 4 has the highest risk while quartile 1 has the lowest risk.

FIG. 3 shows Total Joint Replacement percentage (Y axis) in OA patients from all patients (including all fasinumab, NSAIDs or placebo treated patients) in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 4 shows Total Joint Replacement percentage (Y axis) in OA patients from all fasinumab doses (including all 1q4w, 3q4w and 6q8w) treated patients in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 5 shows Total Joint Replacement percentage (Y axis) in OA patients from fasinumab high doses (including both 3q4w and 6q8w) treated patients in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 6 shows Total Joint Replacement percentage (Y axis) in OA patients from fasinumab 1q4w treated patients in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 7 shows Total Joint Replacement percentage (Y axis) in OA patients from NSAID treated patients in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 8 shows Total Joint Replacement percentage (Y axis) in OA patients from placebo treated patients in R475-OA-1688 clinical trial, stratified by proteomic risk score quartiles (X axis); quartile 4 has the highest risk while quartile 1 has the lowest risk; OR per SD=Odds ratio per standard deviation, N samples=number of subjects in study, N=number of subjects in quartiles.

FIG. 9 shows change of least square mean of WOMAC pain score (Y axis) from baseline over time (X axis) in all fasinumab 1q4w, NSAIDs and placebo treated patients, stratified by proteomic risk score risk groups. The high-risk group is defined by patients in the 4th OA PRS quartile and the 1st to 3rd quartiles of patients are defined as low-risk group.

FIG. 10 shows change of least square mean of WOMAC functional score (Y axis) from baseline over time (X axis) in all fasinumab 1q4w, NSAIDs and placebo treated patients, stratified by proteomic risk score risk groups. The high-risk group is defined by patients in the 4th OA PRS quartile and the 1st to 3rd quartiles of patients are defined as low-risk group.

FIG. 11 shows change of least square mean of WOMAC pain score (Y axis) from baseline over time (X axis) in all fasinumab 1q4w, celecoxib, diclofenac and placebo treated patients, stratified by proteomic risk score risk groups. The high-risk group is defined by patients in the 4th OA PRS quartile and the 1st to 3rd quartiles of patients are defined as low-risk group.

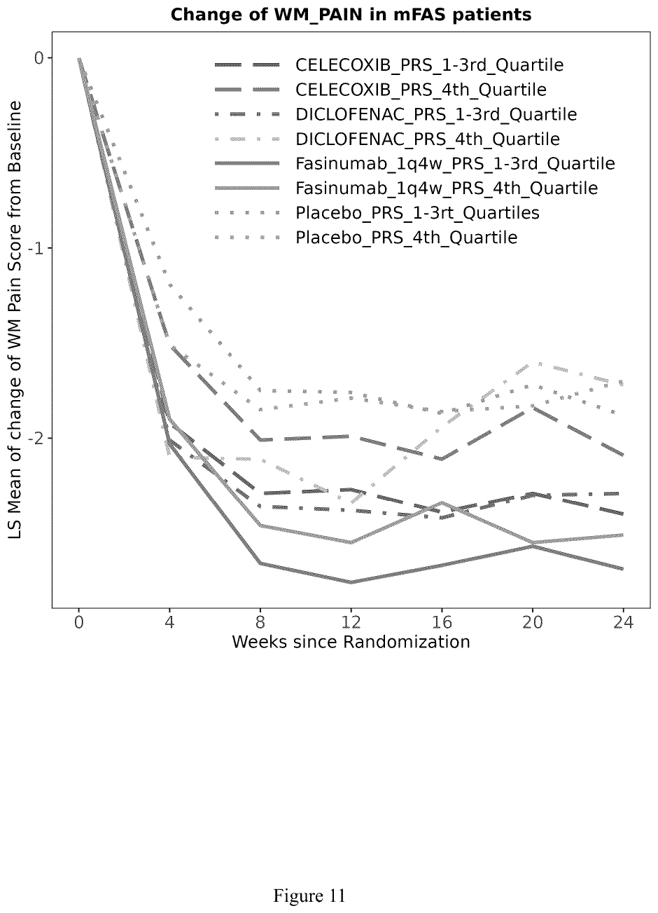

FIG. 12 shows change of least square mean of WOMAC functional score (Y axis) from baseline over time (X axis) in all fasinumab 1q4w, celecoxib, diclofenac and placebo treated patients, stratified by proteomic risk score risk groups. The high-risk group is defined by patients in the 4th OA PRS quartile and the 1st to 3rd quartiles of patients are defined as low-risk group.

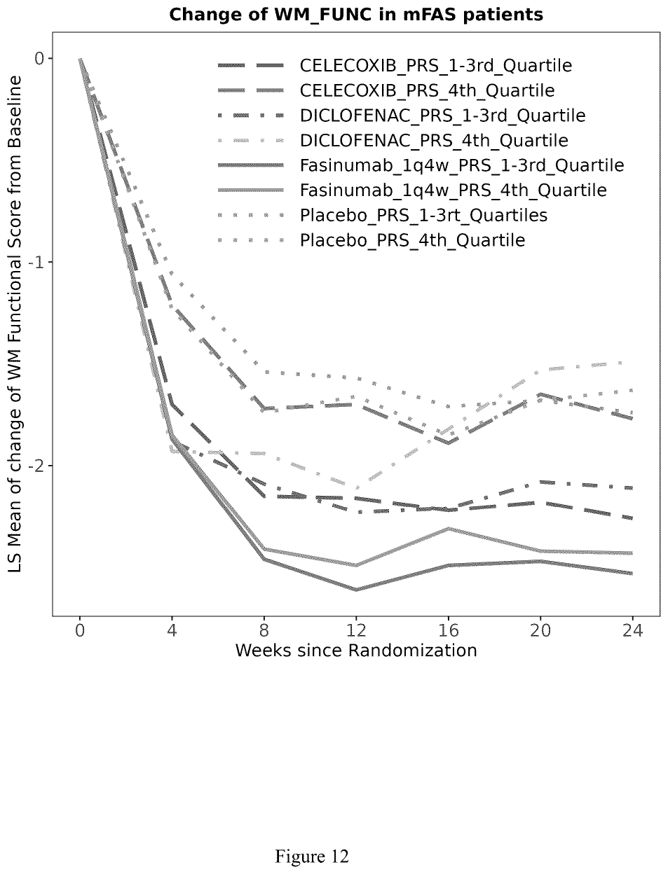

### DESCRIPTION OF EMBODIMENTS

Protein expression levels or circulating levels can play an important role in a risk of developing a disease and potentially influence how such a disease or condition can progress into a more advanced stage requiring particular treatments. Proteomic risk scores combine information from the expression levels or circulating levels of multiple proteins derived from disease association studies to create a single composite quantitative measure for each individual which reflects their disease risk. Risk assessments combining multiple proteins offers the advantage of increased predictive power. An individual with a larger number of relevant protein expression levels or circulating levels for a particular disease will have a higher proteomic risk score than an individual with fewer relevant protein expression levels or circulating levels for the same particular disease. Risk can be evaluated at several thresholds, such as percentiles, standard deviation units of the population distribution, or absolute values. The present disclosure relates generally to the unexpected finding that stratification of subjects by an OA proteomic risk score is useful in the identification of subjects likely to develop OA and/or avoid arthropathic side effects of NGF antagonists in the treatment of OA pain, and to also demonstrate differential response to both NGF antagonists and NSAID treatment.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “antagonist” means either that a given compound is capable of inhibiting the activity of the respective protein or other substance in the cell at least to a certain amount. This can be achieved by a direct interaction of the compound with the given protein or substance (“direct inhibition”) or by an interaction of the compound with other proteins or other substances in or outside the cell which leads to an at least partial inhibition of the activity of the protein or substance (“indirect inhibition”). Inhibition of protein activity can also be achieved through suppressing the expression of a target protein. Techniques of inhibiting protein expression include, but not limited to, antisense inhibition, siRNA-mediated inhibition, miRNA mediated inhibition, ribozyme-mediated inhibition, DNA-directed RNA interference (DdRNAi), RNA-directed DNA methylation, transcription activator-like effector nucleases (TALEN)-mediated inhibition, zinc finger nuclease-mediated inhibition, aptamer-mediated inhibition, and CRISPR-mediated inhibition.

As used herein, the term “nerve growth factor” and “NGF” refers to nerve growth factor and variants (including, for example, splice variants and protein processing variants) thereof that retain at least part of the activity of NGF. As used herein, NGF includes all mammalian species of native sequence NGF, including human, non-human primate, canine, feline, equine, or bovine.

An “NGF antagonist” refers to any molecule that blocks, suppresses, or reduces (including significantly) NGF biological activity, including downstream pathways mediated by NGF signaling, such as receptor binding and/or elicitation of a cellular response to NGF. The term “antagonist” implies no specific mechanism of biological action whatsoever, and is deemed to expressly include and encompass all possible pharmacological, physiological, and biochemical interactions with NGF whether direct or indirect, or whether interacting with NGF, its receptor, or through another mechanism, and its consequences which can be achieved by a variety of different, and chemically divergent, compositions. Exemplary NGF antagonists include, but are not limited to, an anti-NGF antibody, an antisense molecule directed to an NGF (including an antisense molecule directed to a nucleic acid encoding NGF), an NGF antagonist compound, an NGF structural analog, a dominant-negative mutation of a TrkA receptor that binds an NGF, a TrkA immunoadhesin, an anti-TrkA antibody, an anti-p75 antibody, an anti-sense molecule directed to either or both of the TrkA and/or p75 receptors (including anti-sense molecules directed to a nucleic acid molecule encoding TrkA or p75), and a kinase inhibitor. For purpose of the present disclosure, it should be understood that the term “antagonist” encompass all the previously identified terms, titles, and functional states and characteristics whereby the NGF itself, an NGF biological activity (including but not limited to its ability to mediate any aspect of pain), or the consequences of the biological activity, are substantially nullified, decreased, or neutralized in any meaningful degree. In some embodiments, an NGF antagonist binds (physically interact with) NGF (e.g., an antibody), binds to an NGF receptor (such as trkA receptor or p75 receptor), reduces (impedes and/or blocks) downstream NGF receptor signaling, and/or inhibits (reduces) NGF synthesis, production or release. In some embodiments, an NGF antagonist binds (physically interacts with) NGF (e.g., an antibody), binds to an NGF receptor (such as TrkA receptor or p75 receptor), and/or reduces (impedes and/or blocks) downstream NGF receptor signaling. In other embodiments, an NGF antagonist binds NGF and prevents TrkA receptor dimerization and/or TrkA autophosphorylation. In other embodiments, an NGF antagonist inhibits or reduces NGF synthesis and/or production (release). Examples of types of NGF antagonists are provided herein.

As used herein, the term “NSAID” refers to any non-steroidal anti-inflammatory drug including, but not limited to, any of the NSAIDs disclosed herein.

As used herein, “prophylactic treatment” and “prophylaxis” refer to administration of a subject who is not currently nor ever has had OA.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

As used herein, “therapeutic treatment” refers to administering a therapeutic agent to a subject having OA.

As used herein, the terms “treat”, “treatment”, or “treating” refers to administering a therapeutic agent for prophylactic and/or therapeutic purposes.

The present disclosure provides methods of determining the risk of a subject for developing osteoarthritis (OA). The methods comprise determining or having determined the subject's osteoarthritis proteomic risk score, wherein the osteoarthritis proteomic risk score comprises a weighted aggregate of the expression level or circulating level of one or more proteins associated with OA. When the subject's osteoarthritis proteomic risk score is greater than or equal to a threshold osteoarthritis proteomic risk score, the subject has an increased risk of developing OA. When the subject's osteoarthritis proteomic risk score is less than a threshold osteoarthritis proteomic risk score, the subject has a decreased risk of developing OA.

The present disclosure also provides methods of determining the risk of a subject progressing to total joint replacement (TJR). The methods comprise determining or having determined the subject's osteoarthritis proteomic risk score, wherein the osteoarthritis proteomic risk score comprises a weighted aggregate of the expression level or circulating level of one or more proteins associated with OA. When the subject's osteoarthritis proteomic risk score is greater than or equal to a threshold osteoarthritis proteomic risk score, the subject has an increased risk of progressing to TJR. When the subject's osteoarthritis proteomic risk score is less than a threshold osteoarthritis proteomic risk score, the subject has a decreased risk of progressing to TJR.

The present disclosure also provides methods of determining the treatment response of a subject treated with an NGF antagonist, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has less response to an NGF antagonist; and when the subject's osteoarthritis proteomic risk score is less than a threshold osteoarthritis proteomic risk score, the subject has an increased response to an NGF antagonist.

The present disclosure also provides methods of determining the treatment response of a subject treated with an NSAID, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has diminishing response to an NSAID; and when the subject's osteoarthritis proteomic risk score is less than a threshold osteoarthritis proteomic risk score, the subject has an increased response to an NSAID.

The present disclosure also provides methods of determining the treatment response of a subject, the methods comprising: determining or having determined the subject's osteoarthritis proteomic risk score, wherein the osteoarthritis proteomic risk score comprises a weighted aggregate of the expression level or circulating level of one or more proteins associated with OA; wherein: when the subject's osteoarthritis proteomic risk score is greater than or equal to a threshold osteoarthritis proteomic risk score, the subject has less treatment response; and when the subject's osteoarthritis proteomic risk score is less than a threshold osteoarthritis proteomic risk score, the subject has an increase in treatment response.

In some embodiments, a subject may have had OA within the past 1, 2, 3, 4, 5, 6, 7, 8, 9, 10, 11, 12, 13, 14, 15, 16, 17, or 18 months. In some embodiments, a subject may have had OA within the past 1, 2, 3, 4, 5, 6, 7, 8, 9, 10, 11, 12, 13, 14, 15, 16, 17, or 18 years. The subjects may include subjects that have been hospitalized with OA-related symptoms and subjects that are currently hospitalized.

The osteoarthritis proteomic risk score comprises a weighted aggregate of the expression level or circulating level of one or more proteins associated with OA. The number of proteins associated with OA can be 1 protein, at least about 2 proteins, at least about 5 proteins, at least about 10 proteins, at least about 15 proteins, at least about 20 proteins, at least about 30 proteins, at least about 40 proteins, at least about 50 proteins, at least about 100 proteins, or at least about 200 proteins.

Table 1 shows protein wide association analysis with OA discovery data for the top 199 proteins which are statistically significant after Bonferroni Correction (p-value cutoff: 1.7e-5).

TABLE 1
Summary of OA Proteomic Wide Association Analysis

| Protein_names | Effect | Pval |
|---|---|---|
| COL9A1 | 0.8078 | 1.2186E−267 |
| CRTAC1 | 0.8167 | 1.9083E−102 |
| SCRG1 | 0.7353 | 1.3166E−51 |
| ACAN | 0.5014 | 2.9862E−36 |
| CCN3 | 0.4392 | 5.8181E−35 |
| CHGA | 0.1561 | 2.2040E−34 |
| KLK4 | 0.2165 | 3.9357E−34 |
| RBFOX3 | 0.3947 | 1.5001E−33 |
| DCN | 0.5760 | 3.4895E−32 |
| MATN3 | 0.3994 | 4.8049E−31 |
| COMP | 0.3687 | 1.1706E−29 |
| CDCP1 | 0.2331 | 3.4496E−29 |
| BRK1 | 0.2654 | 5.7900E−29 |
| GAST | 0.0939 | 1.4338E−28 |
| COL6A3 | 0.4624 | 2.9468E−27 |
| AMBP | 0.6378 | 9.5969E−27 |
| VSIG2 | 0.1991 | 6.1907E−25 |
| FGFBP2 | 0.2359 | 1.7460E−24 |
| ANXA10 | 0.1250 | 1.4478E−21 |
| CELA2A | −0.1753 | 8.2968E−21 |
| TIMD4 | 0.2333 | 2.3514E−20 |
| SPP1 | 0.2257 | 1.2121E−19 |
| DLK1 | −0.1791 | 2.0346E−18 |
| PGA4 | 0.1420 | 2.3130E−18 |
| ELN | 0.2960 | 4.8731E−18 |
| IGFBP4 | 0.3011 | 9.5697E−18 |
| FABP3 | 0.2149 | 1.1918E−17 |
| TREM2 | 0.1786 | 7.9770E−17 |
| GPNMB | 0.3175 | 1.6294E−16 |
| CYTL1 | −0.4206 | 2.3610E−16 |
| CKAP4 | 0.3788 | 4.0060E−16 |
| CD276 | 0.2551 | 6.6800E−16 |
| EPHA2 | 0.3251 | 6.3196E−15 |
| HSPB6 | 0.2341 | 8.1902E−15 |
| ADGRG2 | 0.3091 | 1.0840E−14 |
| MYOM3 | 0.0963 | 5.3918E−14 |
| LTA4H | 0.1339 | 9.9924E−14 |
| MYBPC1 | 0.1224 | 1.7786E−13 |
| MMP13 | 0.2399 | 3.7676E−13 |
| NCS1 | 0.2787 | 3.9250E−13 |
| CHCHD10 | 0.2214 | 3.9326E−13 |
| MYL3 | 0.1188 | 4.1044E−13 |
| MMP12 | 0.1492 | 4.5913E−13 |
| HAVCR2 | 0.2594 | 5.3012E−13 |
| LMNB2 | 0.3066 | 6.9955E−13 |
| TFF1 | 0.1058 | 7.0044E−13 |
| EDA2R | 0.2498 | 1.3678E−12 |
| NCAN | −0.1979 | 2.4209E−12 |
| ASGR1 | 0.2733 | 6.7360E−12 |
| ACE2 | 0.1376 | 6.8251E−12 |
| C4BPB | 0.1846 | 7.9291E−12 |
| CRELD1 | 0.2134 | 1.6132E−11 |
| CHI3L1 | 0.0926 | 3.4084E−11 |
| NELL2 | −0.2402 | 5.3312E−11 |
| CNTN1 | −0.2491 | 8.2164E−11 |
| BCAT1 | 0.2321 | 8.7354E−11 |
| YAP1 | 0.3182 | 1.5463E−10 |
| CD302 | 0.2639 | 2.3956E−10 |
| LPO | −0.1166 | 2.7927E−10 |
| NCR3LG1 | 0.2232 | 2.8488E−10 |
| MANSC4 | 0.1090 | 2.8601E−10 |
| HS6ST2 | −0.2348 | 3.9222E−10 |
| UMOD | −0.1296 | 4.7976E−10 |
| AREG | 0.1546 | 5.3180E−10 |
| ADAM12 | 0.2117 | 6.8548E−10 |
| IGFBP1 | 0.0557 | 7.1114E−10 |
| SCT | −0.1546 | 8.0760E−10 |
| COLEC12 | 0.3391 | 9.1246E−10 |
| PRELP | 0.3083 | 9.2057E−10 |
| ACTN2 | 0.1205 | 1.0076E−09 |
| OCLN | 0.1704 | 1.8018E−09 |
| LGALS3 | 0.2217 | 1.9192E−09 |
| CTSL | 0.2689 | 2.4926E−09 |
| DPP6 | −0.1882 | 2.7750E−09 |
| TTR | 0.2653 | 2.9102E−09 |
| LIPF | 0.1136 | 3.2612E−09 |
| TSHB | −0.0820 | 3.2814E−09 |
| PLAUR | 0.2624 | 3.2988E−09 |
| CNTN2 | −0.1374 | 3.9157E−09 |
| PSPN | −0.0680 | 4.0133E−09 |
| ARG1 | −0.1183 | 4.4129E−09 |
| SCGB1A1 | −0.1196 | 5.8356E−09 |
| BPIFA2 | −0.0654 | 5.9901E−09 |
| FST | 0.1269 | 6.0983E−09 |
| IGFBP2 | 0.1069 | 6.3180E−09 |
| AGR2 | 0.0648 | 7.0071E−09 |
| TFF2 | 0.1152 | 9.3929E−09 |
| NRCAM | −0.2108 | 9.4726E−09 |
| SUSD5 | 0.2009 | 1.0060E−08 |
| LILRB4 | 0.1591 | 1.0712E−08 |
| IGSF9 | −0.0813 | 1.2023E−08 |
| TNC | 0.1283 | 1.3836E−08 |
| PKLR | −0.0927 | 1.8098E−08 |
| TPK1 | 0.2304 | 1.8459E−08 |
| ULBP2 | 0.1858 | 2.4892E−08 |
| HYOU1 | 0.2888 | 2.5532E−08 |
| DPP10 | −0.1689 | 2.7309E−08 |
| BLVRB | −0.0913 | 2.8450E−08 |
| CA1 | −0.0988 | 2.8961E−08 |
| ART3 | −0.1911 | 2.9770E−08 |
| NELL1 | −0.1451 | 3.3032E−08 |
| MSR1 | 0.1334 | 3.5871E−08 |
| TNFRSF10A | 0.1913 | 4.2323E−08 |
| HAVCR1 | 0.0933 | 5.0114E−08 |
| PRDX2 | −0.1010 | 5.7550E−08 |
| CSPG4 | 0.1904 | 5.8618E−08 |
| GDF15 | 0.1419 | 7.1999E−08 |
| LRRN1 | −0.1500 | 8.1193E−08 |
| TNFRSF12A | 0.1925 | 8.2214E−08 |
| CDHR2 | −0.0817 | 1.0440E−07 |
| PPP3R1 | 0.1389 | 1.0559E−07 |
| TNFRSF9 | 0.1584 | 1.1438E−07 |
| BCAN | −0.1625 | 1.1487E−07 |
| CBLN4 | −0.1685 | 1.2452E−07 |
| CKB | 0.1090 | 1.2738E−07 |
| ADAM9 | 0.2092 | 1.2908E−07 |
| NECTIN2 | 0.2256 | 1.2955E−07 |
| DSCAM | −0.1728 | 1.4204E−07 |
| C9orf40 | −0.0746 | 1.5521E−07 |
| DDI2 | −0.0766 | 1.7160E−07 |
| MMP3 | 0.1183 | 2.3240E−07 |
| ADIPOQ | 0.0886 | 2.5438E−07 |
| ST3GAL1 | 0.1309 | 2.6283E−07 |
| LAYN | 0.1994 | 3.1526E−07 |
| HMBS | −0.1229 | 3.2769E−07 |
| LTBR | 0.2306 | 3.3984E−07 |
| PRCP | −0.1740 | 3.8924E−07 |
| CENPF | −0.1068 | 4.1161E−07 |
| CELA3A | −0.0947 | 4.5221E−07 |
| CA12 | 0.1721 | 4.8149E−07 |
| SLITRK1 | −0.1846 | 5.0389E−07 |
| MYOM2 | 0.1137 | 5.2091E−07 |
| ERBB4 | −0.2131 | 5.5920E−07 |
| SCARB2 | 0.2282 | 6.6198E−07 |
| COL18A1 | 0.2672 | 8.5501E−07 |
| LRP11 | 0.1645 | 8.6837E−07 |
| THBS4 | 0.1167 | 9.1670E−07 |
| PTN | 0.1079 | 9.3150E−07 |
| PHOSPHO1 | 0.1892 | 9.3204E−07 |
| ASGR2 | 0.2314 | 9.5005E−07 |
| RARRES2 | 0.1162 | 1.0678E−06 |
| CCL7 | 0.0902 | 1.2085E−06 |
| STC1 | −0.1270 | 1.2607E−06 |
| BGLAP | 0.0670 | 1.2648E−06 |
| IL15 | 0.1576 | 1.3894E−06 |
| TGM2 | −0.0686 | 1.4386E−06 |
| LTBP2 | 0.1653 | 1.4550E−06 |
| IFI30 | 0.1416 | 1.5271E−06 |
| SPINK5 | −0.1621 | 1.6732E−06 |
| WFIKKN1 | −0.1261 | 1.6862E−06 |
| EPHB4 | 0.2393 | 1.8453E−06 |
| DTNB | 0.1274 | 1.9761E−06 |
| NCF2 | −0.0609 | 1.9919E−06 |
| OGN | 0.1491 | 2.0253E−06 |
| AHSP | −0.0616 | 2.1877E−06 |
| TNFRSF14 | 0.1954 | 2.2173E−06 |
| TCTN3 | 0.1826 | 2.2759E−06 |
| AMPD3 | −0.0756 | 2.5436E−06 |
| TNFRSF11B | 0.1717 | 2.7708E−06 |
| ENAH | 0.1061 | 2.8089E−06 |
| LGALS1 | 0.1577 | 2.9566E−06 |
| HAGH | −0.0683 | 2.9744E−06 |
| FETUB | 0.1566 | 2.9971E−06 |
| FNDC1 | 0.1177 | 3.1235E−06 |
| CLEC5A | 0.1742 | 3.1724E−06 |
| ADAMTS13 | −0.1995 | 3.2606E−06 |
| CSRP3 | 0.0555 | 3.6878E−06 |
| KRT19 | 0.0769 | 3.7511E−06 |
| CRLF1 | −0.1958 | 3.7816E−06 |
| BAG3 | 0.1296 | 3.8531E−06 |
| SOST | 0.1306 | 3.9046E−06 |
| FCRL5 | −0.0875 | 4.1375E−06 |
| ACHE | −0.1436 | 5.1995E−06 |
| ACTA2 | 0.1118 | 6.0286E−06 |
| CCDC80 | 0.1423 | 6.0937E−06 |
| ODAM | −0.0925 | 6.4970E−06 |
| CSTB | 0.1220 | 6.6185E−06 |
| SEZ6L | −0.1811 | 7.0576E−06 |
| STC2 | −0.1797 | 7.2685E−06 |
| MYL4 | −0.1013 | 7.2742E−06 |
| ALDH1A1 | −0.0902 | 7.5143E−06 |
| MXRA8 | 0.1699 | 7.5165E−06 |
| CAPG | 0.0820 | 8.1014E−06 |
| SLC4A1 | −0.1083 | 8.1809E−06 |
| CTRB1 | −0.0872 | 8.5387E−06 |
| TFF3 | 0.1170 | 8.7659E−06 |
| NPPC | −0.1144 | 9.3095E−06 |
| CST5 | −0.0884 | 9.7538E−06 |
| LRP1 | 0.1746 | 9.8062E−06 |
| CCL5 | 0.0476 | 9.8104E−06 |
| CA2 | −0.0675 | 1.1913E−05 |
| PSG1 | 0.0421 | 1.2230E−05 |
| FABP6 | 0.0859 | 1.2537E−05 |
| EFNA1 | 0.2003 | 1.3891E−05 |
| THBD | 0.1815 | 1.4086E−05 |
| DNAJA4 | −0.0692 | 1.4553E−05 |
| IL4R | 0.1303 | 1.4993E−05 |
| ENPP6 | −0.1331 | 1.5634E−05 |
| PTH | −0.0636 | 1.6534E−05 |

In some embodiments, the one or more proteins associated with OA comprises Collagen alpha-1(IX) chain (COL9A1), Cartilage acidic protein 1 (CRTAC1), Scrapie-responsive protein 1 (SCRG1), Aggrecan core protein (ACAN), CCN family member 3 (CCN3), Chromogranin-A (CHGA), Kallikrein-4 (KLK4), RNA binding protein fox-1 homolog 3 (RBFOX3), Decorin (DCN), Matrilin-3 (MATN3), Cartilage oligomeric matrix protein (COMP), CUB domain-containing protein 1 (CDCP1), Protein BRICK1 (BRK1), Gastrin (GAST), Collagen alpha-3(VI) chain (COL6A3), Protein AMBP (AMBP), V-set and immunoglobulin domain-containing protein 2 (VSIG2), Fibroblast growth factor-binding protein 2 (FGFBP2), Annexin A10 (ANXA10), Chymotrypsin-like elastase family member 2A (CELA2A), T-cell immunoglobulin and mucin domain-containing protein 4 (TIMD4), Osteopontin (SPP1), Protein delta homolog 1 (DLK1), Pepsin A-4 (PGA4), Elastin (ELN), Insulin-like growth factor-binding protein 4 (IGFBP4), Fatty acid-binding protein, heart (FABP3), Triggering receptor expressed on myeloid cells 2 (TREM2), Transmembrane glycoprotein NMB (GPNMB), Cytokine-like protein 1 (CYTL1), Cytoskeleton-associated protein 4 (CKAP4), CD276 antigen (CD276), Ephrin type-A receptor 2 (EPHA2), Heat shock protein beta-6 (HSPB6), Adhesion G-protein coupled receptor G2 (ADGRG2), Myomesin-3 (MYOM3), Leukotriene A-4 hydrolase (LTA4H), Myosin-binding protein C, slow-type (MYBPC1), Collagenase 3 (MMP13), Neuronal calcium sensor 1 (NCS1), Coiled-coil-helix-coiled-coil-helix domain-containing protein 10, mitochondrial (CHCHD10), Myosin light chain 3 (MYL3), Macrophage metalloelastase (MMP12), Hepatitis A virus cellular receptor 2 (HAVCR2), Lamin-B2 (LMNB2), Trefoil factor 1 (TFF1), Tumor necrosis factor receptor superfamily member 27 (EDA2R), Neurocan core protein (NCAN), Asialoglycoprotein receptor 1 (ASGR1), Angiotensin-converting enzyme 2 (ACE2), C4b-binding protein beta chain (C4BPB), Protein disulfide isomerase CRELD1 (CRELD1), Chitinase-3-like protein 1 (CHI3L1), Protein kinase C-binding protein NELL2 (NELL2), Contactin-1 (CNTN1), Branched-chain-amino-acid aminotransferase, cytosolic (BCAT1), Transcriptional coactivator YAP1 (YAP1), CD302 antigen (CD302), Lactoperoxidase (LPO), Natural cytotoxicity triggering receptor 3 ligand 1 (NCR3LG1), MANSC domain-containing protein 4 (MANSC4), Heparan-sulfate 6-O-sulfotransferase 2 (HS6ST2), Uromodulin (UMOD), Amphiregulin (AREG), Disintegrin and metalloproteinase domain-containing protein 12 (ADAM12), Insulin-like growth factor-binding protein 1 (IGFBP1), Secretin (SCT), Collectin-12 (COLEC12), Prolargin (PRELP), Alpha-actinin-2 (ACTN2), Occludin (OCLN), Galectin-3 (LGALS3), Cathepsin L1 (CTSL), Dipeptidyl aminopeptidase-like protein 6 (DPP6), Transthyretin (TTR), Gastric triacylglycerol lipase (LIPF), Thyrotropin subunit beta (TSHB), Urokinase plasminogen activator surface receptor (PLAUR), Contactin-2 (CNTN2), Persephin (PSPN), Arginase-1 (ARG1), Uteroglobin (SCGB1A1), BPI fold-containing family A member 2 (BPIFA2), Follistatin (FST), Insulin-like growth factor-binding protein 2 (IGFBP2), Anterior gradient protein 2 homolog (AGR2), Trefoil factor 2 (TFF2), Neuronal cell adhesion molecule (NRCAM), Sushi domain-containing protein 5 (SUSD5), Leukocyte immunoglobulin-like receptor subfamily B member 4 (LILRB4), Protein turtle homolog A (IGSF9), Tenascin (TNC), Pyruvate kinase PKLR (PKLR), Thiamin pyrophosphokinase 1 (TPK1), UL16-binding protein 2 (ULBP2), Hypoxia up-regulated protein 1 (HYOU1), Inactive dipeptidyl peptidase 10 (DPP10), Flavin reductase (NADPH) (BLVRB), Carbonic anhydrase 1 (CA1), Ecto-ADP-ribosyltransferase 3 (ART3), Protein kinase C-binding protein NELL1 (NELL1), Macrophage scavenger receptor types I and II (MSR1), Tumor necrosis factor receptor superfamily member 10A (TNFRSF10A), Hepatitis A virus cellular receptor 1 (HAVCR1), Peroxiredoxin-2 (PRDX2), Chondroitin sulfate proteoglycan 4 (CSPG4), Growth/differentiation factor 15 (GDF15), Leucine-rich repeat neuronal protein 1 (LRRN1), Tumor necrosis factor receptor superfamily member 12A (TNFRSF12A), Cadherin-related family member 2 (CDHR2), Calcineurin subunit B type 1 (PPP3R1), Tumor necrosis factor receptor superfamily member 9 (TNFRSF9), Brevican core protein (BCAN), Cerebellin-4 (CBLN4), Creatine kinase B-type (CKB), Disintegrin and metalloproteinase domain-containing protein 9 (ADAM9), Nectin-2 (NECTIN2), Down syndrome cell adhesion molecule (DSCAM), Uncharacterized protein C9orf40 (C9orf40), Protein DDI1 homolog 2 (DDI2), Stromelysin-1 (MMP3), Adiponectin (ADIPOQ), CMP-N-acetylneuraminate-beta-galactosamide-alpha-2,3-sialyltransferase 1 (ST3GAL1), Layilin (LAYN), Porphobilinogen deaminase (HMBS), Tumor necrosis factor receptor superfamily member 3 (LTBR), Lysosomal Pro-X carboxypeptidase (PRCP), Centromere protein F (CENPF), Chymotrypsin-like elastase family member 3A (CELA3A), Carbonic anhydrase 12 (CA12), SLIT and NTRK-like protein 1 (SLITRK1), Myomesin-2 (MYOM2), Receptor tyrosine-protein kinase erbB-4 (ERBB4), Lysosome membrane protein 2 (SCARB2), Collagen alpha-1(XVIII) chain (COL18A1), Low-density lipoprotein receptor-related protein 11 (LRP11), Thrombospondin-4 (THBS4), Pleiotrophin (PTN), Phosphoethanolamine/phosphocholine phosphatase (PHOSPHO1), Asialoglycoprotein receptor 2 (ASGR2), Retinoic acid receptor responder protein 2 (RARRES2), C-C motif chemokine 7 (CCL7), Stanniocalcin-1 (STC1), Osteocalcin (BGLAP), Interleukin-15 (IL15), Protein-glutamine gamma-glutamyltransferase 2 (TGM2), Latent-transforming growth factor beta-binding protein 2 (LTBP2), Gamma-interferon-inducible lysosomal thiol reductase (IFI30), Serine protease inhibitor Kazal-type 5 (SPINK5), WAP, Kazal, immunoglobulin, Kunitz and NTR domain-containing protein 1 (WFIKKN1), Ephrin type-B receptor 4 (EPHB4), Dystrobrevin beta (DTNB), Neutrophil cytosol factor 2 (NCF2), Mimecan (OGN), Alpha-hemoglobin-stabilizing protein (AHSP), Tumor necrosis factor receptor superfamily member 14 (TNFRSF14), Tectonic-3 (TCTN3), AMP deaminase 3 (AMPD3), Tumor necrosis factor receptor superfamily member 11B (TNFRSF11B), Protein enabled homolog (ENAH), Galectin-1 (LGALS1), Hydroxyacylglutathione hydrolase, mitochondrial (HAGH), Fetuin-B (FETUB), Fibronectin type III domain-containing protein 1 (FNDC1), C-type lectin domain family 5 member A (CLEC5A), A disintegrin and metalloproteinase with thrombospondin motifs 13 (ADAMTS13), Cysteine and glycine-rich protein 3 (CSRP3), Keratin, type I cytoskeletal 19 (KRT19), Cytokine receptor-like factor 1 (CRLF1), BAG family molecular chaperone regulator 3 (BAG3), Sclerostin (SOST), Fc receptor-like protein 5 (FCRL5), Acetylcholinesterase (ACHE), Actin, aortic smooth muscle (ACTA2), Coiled-coil domain-containing protein 80 (CCDC80), Odontogenic ameloblast-associated protein (ODAM), Cystatin-B (CSTB), Seizure 6-like protein (SEZ6L), Stanniocalcin-2 (STC2), Myosin light chain 4 (MYL4), Retinal dehydrogenase 1 (ALDH1A1), Matrix remodeling-associated protein 8 (MXRA8), Macrophage-capping protein (CAPG), Band 3 anion transport protein (SLC4A1), Chymotrypsinogen B (CTRB1), Trefoil factor 3 (TFF3), C-type natriuretic peptide (NPPC), Cystatin-D (CST5), Prolow-density lipoprotein receptor-related protein 1 (LRP1), C-C motif chemokine 5 (CCL5), Carbonic anhydrase 2 (CA2), Pregnancy-specific beta-1-glycoprotein 1 (PSG1), Gastrotropin (FABP6), Ephrin-A1 (EFNA1), Thrombomodulin (THBD), DnaJ homolog subfamily A member 4 (DNAJA4), Interleukin-4 receptor subunit alpha (IL4R), Glycerophosphocholine cholinephosphodiesterase ENPP6 (ENPP6), or Parathyroid hormone (PTH), or any combination thereof comprising at least 2, 3, 4, 5, 6, 7, 8, 9, or 10 proteins. In some embodiments, the one or more proteins associated with OA comprises COL9A1, CRTAC1, CNTN1, GAST, CYTL1, KLK4, DLK1, CDCP1, DPP10, CELA2A, RBFOX3, CRELD1, NELL2, PGA4, ARG1, ART3, TIMD4, FGFBP2, BPIFA2, PTH, and/or BRK1, or any combination thereof.

The expression level or circulating level of any one or more of these proteins can be determined by any methodology used for determining protein expression levels or circulating levels. In some embodiments, the protein expression level or circulating level is determined by a proximity extension assay using oligo-conjugated antibody pairs to be measured by sequencing (see, Sun et al., 2022). Other methods include High-Performance Liquid Chromatography (HPLC), Liquid Chromatography-Mass Spectrometry (LC/MS), Enzyme-Linked Immunosorbent Assay (ELISA), protein immunoprecipitation, immunoelectrophoresis, western blot, protein immunostaining. High throughput proteomics include protein pathway arrays, next-generation tissue microarrays, single-cell proteomics, single-molecule proteomics, suspension bead array technology (Luminex), multiplex bead assays (Simoa), nanoparticles (Seer), and proximity extension assay (Olink) Proteomics, aptamer-based multiplexed proteomics (Somalogic) (see, for example, Cui et al., Lab. Invest., 2022, 102, 1170-1181; and Suhre et al., Nat. Rev. Genet., 2021, 22, 19-37).

In some embodiments, the osteoarthritis proteomic risk score is determined by the formula: (0.76×COL9A1 protein level)+(0.67×CRTAC1 protein level)+(−0.26×CNTN1 protein level)+(0.08×GAST protein level)+(−0.45×CYTL1 protein level)+(0.13×KLK4 protein level)+(−0.19×DLK1 protein level)+(0.15×CDCP1 protein level)+(−0.19×DPP10 protein level)+(−0.19×CELA2A protein level)+(0.22×RBFOX3 protein level)+(0.18×CRELD1 protein level)+(−0.23×NELL2 protein level)+(0.10×PGA4 protein level)+(−0.10×ARG1 protein level)+(−0.25×ART3 protein level)+(0.14×TIMD4 protein level)+(0.15×FGFBP2 protein level)+(−0.06×BPIFA2 protein level)+(−0.08×PTH protein level)+(0.12×BRK1 protein level).

In some embodiments, the osteoarthritis proteomic risk score is determined by the formula: (about 0.76×COL9A1 protein level)+(about 0.67×CRTAC1 protein level)+(about −0.26×CNTN1 protein level)+(about 0.08×GAST protein level)+(about −0.45×CYTL1 protein level)+(about 0.13×KLK4 protein level)+(about −0.19×DLK1 protein level)+(about 0.15×CDCP1 protein level)+(about −0.19×DPP10 protein level)+(about −0.19×CELA2A protein level)+(about 0.22×RBFOX3 protein level)+(about 0.18×CRELD1 protein level)+(about −0.23×NELL2 protein level)+(about 0.10×PGA4 protein level)+(about −0.10×ARG1 protein level)+(about −0.25×ART3 protein level)+(about 0.14×TIMD4 protein level)+(about 0.15×FGFBP2 protein level)+(about −0.06×BPIFA2 protein level)+(about −0.08×PTH protein level)+(about 0.12×BRK1 protein level).

In some embodiments, the osteoarthritis proteomic risk score is determined by the formula: (about 0.8×COL9A1 protein level)+(about 0.7×CRTAC1 protein level)+(about −0.3×CNTN1 protein level)+(about 0.1×GAST protein level)+(about −0.4×CYTL1 protein level)+(about 0.1×KLK4 protein level)+(about −0.2×DLK1 protein level)+(about 0.1×CDCP1 protein level)+(about −0.2×DPP10 protein level)+(about −0.2×CELA2A protein level)+(about 0.2×RBFOX3 protein level)+(about 0.2×CRELD1 protein level)+(about −0.2×NELL2 protein level)+(about 0.1×PGA4 protein level)+(about −0.1×ARG1 protein level)+(about −0.2×ART3 protein level)+(about 0.1×TIMD4 protein level)+(about 0.2×FGFBP2 protein level)+(about −0.1×BPIFA2 protein level)+(about −0.1×PTH protein level)+(about 0.1×BRK1 protein level).

The osteoarthritis proteomic risk score is determined from a biological sample obtained from the subject. In some embodiments, the methods described herein further comprises an initial step of obtaining a biological sample from the subject. The biological sample may contain whole cells, live cells and/or cell debris. The biological sample may contain (or be derived from) a bodily fluid. The present disclosure encompasses embodiments wherein the bodily fluid is selected from amniotic fluid, aqueous humor, vitreous humor, bile, blood, serum, plasma, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammalian organism, for example by venipuncture, or other collecting or sampling procedures. In some embodiments, the biological sample comprises blood, serum, plasma, semen, saliva, urine, feces, hair, teeth, bone, tissue, a swab from a cheek, or a cell.

In some embodiments, the threshold osteoarthritis proteomic risk score is generated from a reference population, wherein members of the reference population have OA. In some embodiments, the reference population comprises at least about 100 subjects. In some embodiments, the reference population comprises at least about 200 subjects. In some embodiments, the reference population comprises at least about 500 subjects. In some embodiments, the reference population comprises at least about 1,000 subjects. In some embodiments, the reference population comprises at least about 3,000 subjects. In some embodiments, the reference population comprises at least about 5,000 subjects. In some embodiments, the reference population comprises at least about 7,500 subjects. In some embodiments, the reference population comprises at least about 10,000 subjects. In some embodiments, the reference population comprises at least about 12,000 subjects. In some embodiments, the reference population comprises at least about 15,000 subjects. In some embodiments, the reference population comprises at least about 20,000 subjects. In some embodiments, the reference population comprises at least about 30,000 subjects. In some embodiments, the reference population comprises at least about 50,000 subjects. In some embodiments, the reference population comprises at least about 70,000 subjects. In some embodiments, the reference population comprises at least about 100,000 subjects.

The threshold osteoarthritis proteomic risk score can be determined by a hierarchy. In some embodiments, the hierarchy can be by percentiles. By way of a non-limiting example, the osteoarthritis proteomic risk score may be divided into quintiles, e.g., a top quintile, a top-intermediate quintile, an intermediate quintile, an intermediate-bottom quintile, and a bottom quintile, wherein the top quintile of osteoarthritis proteomic risk score correspond the highest risk group and the bottom quintile of osteoarthritis proteomic risk score correspond to the lowest risk group.

In some embodiments, the threshold osteoarthritis proteomic risk score comprises the highest weighted osteoarthritis proteomic risk scores, including, but not limited to the top 50%, 55%, 60%, 70%, 80%, 90%, or 95% of osteoarthritis proteomic risk score from a subject population. In some embodiments, the threshold osteoarthritis proteomic risk score is a value within the top 50%, 55%, 60%, 70%, 80%, 90%, or 95% percentile. In some embodiments, the threshold osteoarthritis proteomic risk score is a value within the top 50% percentile. In some embodiments, the threshold osteoarthritis proteomic risk score is a value within the top 55% percentile. In some embodiments, the threshold osteoarthritis proteomic risk score is a value within the top 60% percentile. In some embodiments, the threshold osteoarthritis proteomic risk score is a value within the top 65% percentile. In some embodiments, the threshold osteoarthritis proteomic risk score is a value within the top 70% percentile. In some embodiments, the threshold proteomic risk score is a value within the top 75% percentile. In some embodiments, the threshold proteomic risk score is a value within the top 80% percentile. In some embodiments, the threshold proteomic risk score is a value within the top 85% percentile. In some embodiments, the threshold proteomic risk score is a value within the top 90% percentile. In some embodiments, the threshold PRS is a value within the top 95% percentile. In some embodiments, the threshold osteoarthritis proteomic risk score is the top 75% within a reference population. In some embodiments, the threshold osteoarthritis proteomic risk score is the top quintile within a reference population.

Any of the methods described herein can further comprise administering an NGF antagonist and/or a therapeutic agent that treats OA to the subject when the subject's osteoarthritis proteomics risk score is less than or equal to a threshold osteoarthritis proteomics risk score. In some embodiments, the NGF antagonist comprises an antibody, a bispecific antibody, a polypeptide, an antisense nucleic acid molecule, an siRNA molecule, or a small molecule.

Anti-NGF antibodies may bind to NGF and inhibit NGF biological activity and/or downstream pathway(s) mediated by NGF signaling. Numerous anti-NGF antibodies are described in, for example, PCT Publication Nos. WO 00/073344, WO 02/096458, WO 01/78698, and WO 01/64247, U.S. Application Publication No. US2011/0206682; U.S. Pat. Nos. 5,844,092, 5,877,016, and 6,153,189; Hongo et al., Hybridoma, 2000, 19, 215-227; Cell. Molec. Biol., 1993, 13, 559-568; GenBank Accession Nos. U39608, U39609, L17078, and L17077. In some embodiments, the antibody is ABT-110, fasinumab, tanezumab, MEDI7352, or fulranumab. In some embodiments, the antibody is fasinumab. In some embodiments, the antibody is tanezumab. In some embodiments, the antibody is fulranumab. In some embodiments, the antibody is ABT-110.

Antibodies are intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding fragment thereof) may be identical to the human germline sequences, or may be naturally or artificially modified. An amino acid consensus sequence may be defined based on a side-by-side analysis of two or more CDRs.

Antibodies include antigen-binding fragments of full antibody molecules. An antigen-binding portion of an antibody or an antigen-binding fragment of an antibody include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Suitable NGF antagonist polypeptides include, but not limited to, NGF mimetic peptides, which competitively bind TrkA or P75^{NTR} receptors. Numerous NGF antagonist peptides are described, for example, in LeSauteur et al., J. Biol. Chem., 1995, 270, 6564-6569; and Brahimi et al., Biochim. Biophys. Acta, 2010, 1800, 1018-1026; and Longo et al., J. Neurosci. Res., 1997, 48, 1-17; PCT Publication Nos. WO 97/15593 and WO89/09225; and U.S. Pat. Nos. 6,291,247; and 6,017,878. An additional NGF antagonist is MEDI7352.

Antisense inhibition includes reduction of target nucleic acid levels in the presence of an oligonucleotide complementary to a target nucleic acid compared to target nucleic acid levels in the absence of the oligonucleotide.

Suitable small molecule NGF antagonists are described in, for example in U.S. Publication No. 20010046959. Compounds that inhibit NGF's binding to p75 are described in PCT Publication No. WO 00/69829. Compounds that inhibit NGF's binding to TrkA/p75 are described in PCT Publication No. WO 98/17278. Additional examples of NGF antagonists include the compounds described in PCT Publication Nos. WO 02/17914 and WO 02/20479, U.S. Pat. Nos. 5,342,942, 6,127,401, and 6,359,130. Further exemplary NGF antagonists are compounds that are competitive inhibitors of NGF. See U.S. Pat. No. 6,291,247. In some embodiments, the small molecule is K252a, ALE-0540, PQC-083, PD-90780, LM11A-31 dihydrochloride, Y1036, GZ389988A, or Ro 08-2750. In some embodiments, the small molecule is K252a. In some embodiments, the small molecule is ALE-0540. In some embodiments, the small molecule is PQC-083. In some embodiments, the small molecule is PD-90780. In some embodiments, the small molecule is LM11A-31 dihydrochloride. In some embodiments, the small molecule is Y1036. In some embodiments, the small molecule is GZ389988A. In some embodiments, the small molecule is Ro 08-2750.

Any of the methods described herein can further comprise administering an NSAID and/or a therapeutic agent that treats OA to the subject when the subject's osteoarthritis proteomics risk score is less than or equal to a threshold osteoarthritis proteomics risk score. Exemplary NSAIDs include, but are not limited to, ibuprofen, naproxen, diclofenac, etodolac, meloxicam, oxaprozin, celecoxib, piroxicam, indomethacin, or sulindac, or any combination thereof.

Any of the methods described herein can further comprise administering an opioid and/or a therapeutic agent that treats OA to the subject when the subject's osteoarthritis proteomics risk score is less than or equal to a threshold osteoarthritis proteomics risk score. Exemplary opioids include, but are not limited to, codeine, hydrocodone, oxycodone, or tramadol, or any combination thereof.

Any of the methods described herein can further comprise administering an NaV1.8, NaV1.7 inhibitor and/or a therapeutic agent that treats OA to the subject when the subject's osteoarthritis proteomics risk score is less than or equal to a threshold osteoarthritis proteomics risk score. Exemplary NaV 1.8 and/or Nav 1.7 inhibitors include, but are not limited to, VX-548.

In some embodiments, the therapeutic agent that treats OA can comprise an analgesic, a steroid injection, a therapeutic injection, an antidepressant, physical therapy, a strengthening exercise, radiofrequency nerve ablation, or surgery, or any combination thereof.

Examples of analgesics useful for treating OA include, but are not limited to, nonsteroidal anti-inflammatory drugs (NSAIDs) (such as ibuprofen, naproxen, diclofenac, etodolac, meloxicam, oxaprozin, celecoxib, piroxicam, indomethacin, and sulindac), acetaminophen, glucosamine, chondroitin, and opioids (such as codeine, hydrocodone, oxycodone, or tramadol), or any combination thereof.

Examples of steroid injections useful for treating OA include, but are not limited, to corticosteroids (such as triamcinolone, cortisone, prednisone, and methylprednisolone, or any combination thereof).

Examples of therapeutic injections useful for treating OA include, but are not limited to, a hyaluronic acid injection.

Examples of antidepressants useful for treating OA include, but are not limited to, duloxetine, amitriptyline, desipramine, and nortriptyline, or any combination thereof.

In some embodiments, treatment of OA can include physical therapy, cognitive behavioral therapy, and/or weight loss.

An effective amount is an amount sufficient to effect beneficial or desired clinical results including alleviation or reduction in the pain sensation. For purposes of the present disclosure, an effective amount of an NGF antagonist (such as an anti-NGF antibody) includes an amount sufficient to treat, ameliorate, reduce the intensity of or prevent pain (including nociception and the sensation of pain) of any sort, including acute, chronic, inflammatory, neuropathic, or post-surgical pain. In some embodiments, an effective amount of an NGF antagonist is a quantity of the NGF antagonist capable of modulating the sensitivity threshold to external stimuli to a level comparable to that observed in healthy subjects. In some embodiments, this level may not be comparable to that observed in healthy subjects, but is reduced compared to not receiving the combination therapy. As is understood in the art, an effective amount of NGF antagonist may vary, depending on, inter alia, type of pain (and patient history as well as other factors such as the type (and/or dosage) or NGF antagonist used.

In the context of the methods disclosed herein, additional therapeutically active component(s), e.g., any of the agents listed above or derivatives thereof, may be administered just prior to, concurrent with, or shortly after the administration of an NGF antagonist; (for purposes of the present disclosure, such administration regimens are considered the administration of an NGF antagonist “in combination with” an additional therapeutically active component). In some embodiments, an additional therapeutically active component is considered administered “in combination with” an NGF antagonist notwithstanding the fact that the additional therapeutically active component and the NGF antagonist are administered by different routes. The present methods include pharmaceutical compositions and methods of use thereof in which an NGF antagonist is co-formulated with one or more of the additional therapeutically active component(s) as described herein.

Any of the methods described herein can be used to select a population of subjects or candidates for clinical trials, e.g., a clinical trial whose patient population is suitable for treatment by NGF antagonist or other OA or pain (such as chronic pain) treatment regimen. In some embodiments, the selected candidates or subjects are divided into subgroups based on their OA proteomic risk score for each subject or candidate, and the method is used to determine whether a subject has an increased risk of developing OA, progressing to TJR, or having differential pain response. In some embodiments the subjects are selected based on OA proteomic risk score alone. For example, if a subject or a candidate that has an OA proteomic risk score greater than or equal to a threshold, the subject is selected for initiating treatment or a candidate is included in the clinical trial.

In addition, besides stepwise variable selection, other statistical or machine learning algorithms including LASSO, Elastic Net, neural network, and decision tree-based models such as random forest and XGBoost can also be applied to construct proteomic risk score (see, Tibshirani, J. Royal Statistical Society, Series B (Methodological), 1996, 58, 267-88; Zou et al., J. Royal Statistical Society, Series B (Statistical Methodology), 2005, 67, 301-320; Castellano et al., Neurocomputing, 2000, 31, 1-13; Ooka et al., BMJ Nutrition, Prevention & Health, 2021, 4, 140-148; and Yan et al., Ann. Transl. Med., 2022, 10, 860).

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric. It should be understood that these examples are for illustrative purposes only and are not to be construed as limiting the claimed subject matter in any manner.

### EXAMPLES

### Example 1: Proteomic Risk Score Analysis

The Proteomic Risk Score analysis was conducted by using serum proteomic data from UK Biobank (UKB) Pharma Proteomics Project (PPP), referred to as UKB PPP (Sun et al., bioRxiv, 2022.06.17.496443). A total of 46,431 individuals and 2,939 proteins from the UKB PPP dataset passed quality control and were available for analysis.

During the initial run of model building in the training data, OA status was used as a response variable and the proteomic data of each protein as a predictor with the presence of Age, Age Squared (AgeSq), Gender, body mass index (BMI), Osteoarthritis Polygenic Risk Score (OA-PRS), and principal components of genetic data as covariates. To account for multiple testing, a Bonferroni correction was utilized to define a statistical significance cutoff p=1.7e-5 (0.05/2939). After the initial run of the screen, only proteins that have strong association with OA (p-value<1.7e-5) were included as the updated protein candidates. Subsequently, the following two steps were repeated until no proteins were left in the protein candidate pools to generate the final selected proteins.

Step 1: Among the updated protein candidates, the proteins with the strongest association with OA (minimum p-value) were selected. Subsequently, the correlation between the selected protein and the rest of update protein candidates was determined. If there are some proteins that have a high correlation (>0.8) with the selected protein, these proteins were removed from the updated protein candidates. Step 2: The selected protein was added to the covariates and removed from the protein candidates. Steps 1 and 2 were repeated until there were no longer any proteins meeting the minimum statistical threshold. Given the update proteins and covariates list, the association between the proteins remaining in the candidate pools with OA status can be continuously tested.

TABLE 2
Model Fitting in UKB PPP Training Data

| Variable | Estimate | Std. Error | Pr(>\|z\|) |
|---|---|---|---|
| Age | 1.91994097 | 0.2144004 | 3.40E−19 |
| Sex | −0.2218857 | 0.22810494 | 0.33068499 |
| BMI | 0.32065628 | 0.0145479 | 1.15E−107 |
| AgeSq | −1.5223126 | 0.2071316 | 1.99E−13 |
| AgexSex | 0.20493956 | 0.10855348 | 0.05903758 |
| PC1 | 0.01380307 | 0.19123616 | 0.94246014 |
| PC2 | −0.0037852 | 0.15801908 | 0.98088945 |
| PC3 | −0.0799999 | 0.10442652 | 0.44362378 |
| PC4 | 0.02303681 | 0.03232742 | 0.47608763 |
| PC5 | −0.0104896 | 0.01373863 | 0.445157 |
| PC6 | −0.0100957 | 0.03508781 | 0.77355704 |
| PC7 | 0.00632771 | 0.01618259 | 0.69578278 |
| PC8 | −0.0045382 | 0.02234129 | 0.83903377 |
| PC9 | 0.04861344 | 0.01280632 | 0.00014702 |
| PC10 | 0.07535119 | 0.02254372 | 0.00083043 |
| Creatinine | −0.0773666 | 0.09716065 | 0.4258724 |
| Cystatin | −0.1005378 | 0.1136293 | 0.376271 |
| gtarray | 0.00318789 | 0.04037278 | 0.93706342 |
| CurrentSmoke | 0.04554445 | 0.04535462 | 0.31528927 |
| GRS | 0.09588851 | 0.01270977 | 4.54E−14 |
| COL9A1 | 0.7601528 | 0.02417802 | 5.78E−217 |
| CRTAC1 | 0.67423609 | 0.04026019 | 5.96E−63 |
| CNTN1 | −0.2599759 | 0.04529719 | 9.50E−09 |
| GAST | 0.07522688 | 0.00932635 | 7.26E−16 |
| CYTL1 | −0.4451532 | 0.05462235 | 3.65E−16 |
| KLK4 | 0.13480271 | 0.01870138 | 5.67E−13 |
| DLK1 | −0.1902313 | 0.02313499 | 1.99E−16 |
| CDCP1 | 0.14794426 | 0.02307813 | 1.45E−10 |
| DPP10 | −0.1938539 | 0.03536193 | 4.21E−08 |
| CELA2A | −0.1895557 | 0.02004604 | 3.20E−21 |
| RBFOX3 | 0.222883 | 0.03588024 | 5.24E−10 |
| CRELD1 | 0.1824498 | 0.0342222 | 9.75E−08 |
| NELL2 | −0.2270052 | 0.04432088 | 3.03E−07 |
| PGA4 | 0.09922422 | 0.01702408 | 5.59E−09 |
| ARG1 | −0.0998823 | 0.0213272 | 2.82E−06 |
| ART3 | −0.247908 | 0.03993116 | 5.35E−10 |
| TIMD4 | 0.14153908 | 0.02723314 | 2.02E−07 |
| FGFBP2 | 0.15113661 | 0.02483708 | 1.16E−09 |
| BPIFA2 | −0.0637089 | 0.01178316 | 6.42E−08 |
| PTH | −0.0812131 | 0.0155355 | 1.72E−07 |
| BRK1 | 0.11939352 | 0.02554946 | 2.97E−06 |

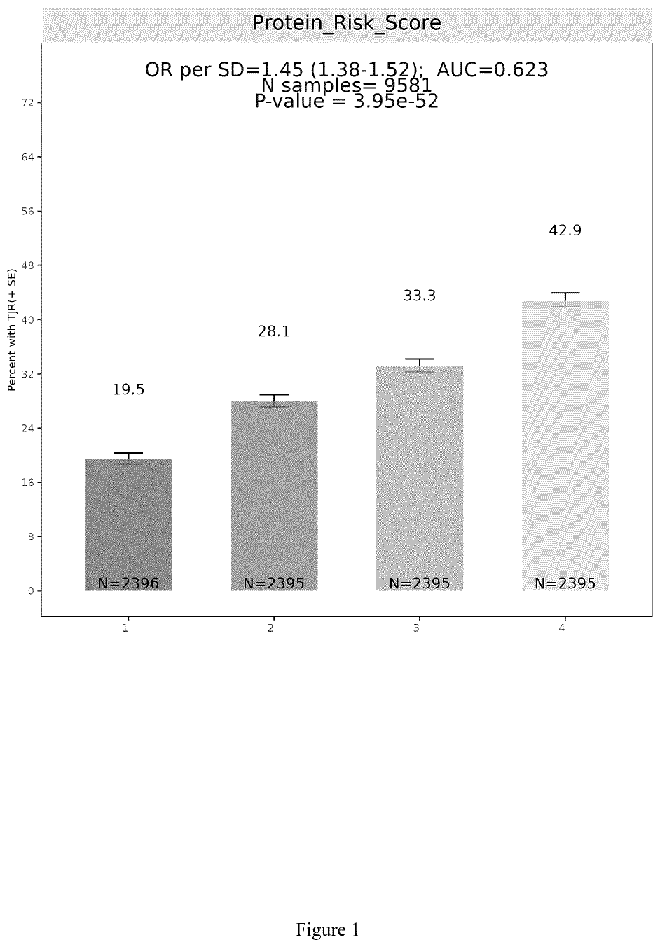

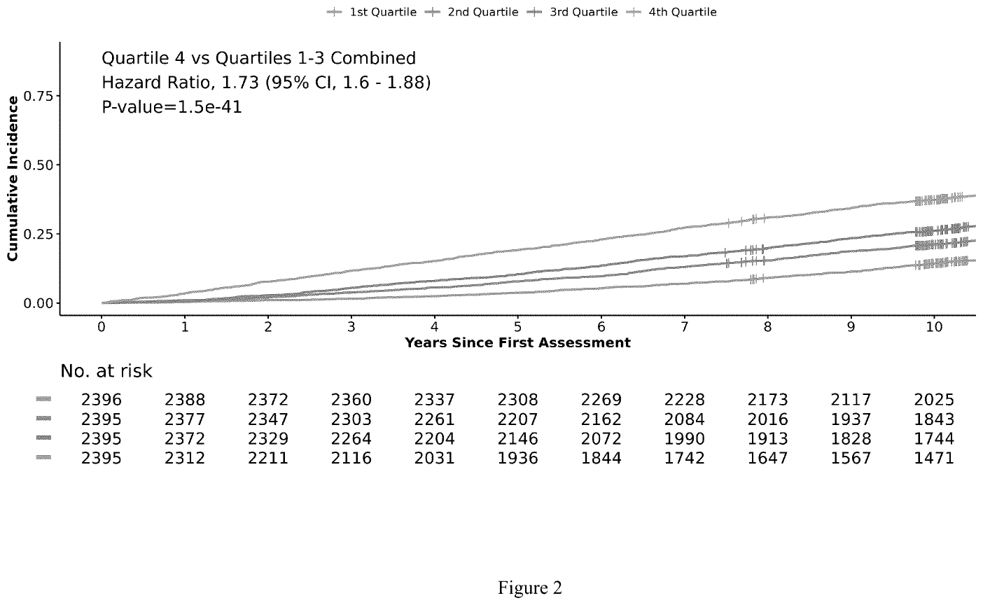

After all the proteins were selected, the prediction models were refitted in the discovery data set as shown in Table 2. The final OA proteomic score can be calculated as follows: (0.760153×COL9A1 protein level)+(0.674236×CRTAC1 protein level)+(−0.259976×CNTN1 protein level)+(0.075227×GAST protein level)+(−0.445153×CYTL1 protein level)+(0.134803×KLK4 protein level)+(−0.190231×DLK1 protein level)+(0.147944×CDCP1 protein level)+(−0.193854×DPP10 protein level)+(−0.189556×CELA2A protein level)+(0.222883×RBFOX3 protein level)+(0.182450×CRELD1 protein level)+(−0.227005×NELL2 protein level)+(0.099224×PGA4 protein level)+(−0.099882×ARG1 protein level)+(−0.247908×ART3 protein level)+(0.141539×TIMD4 protein level)+(0.151137×FGFBP2 protein level)+(−0.063709×BPIFA2 protein level)+(−0.081213×PTH protein level)+(0.119394×BRK1 protein level). Then the total joint replacement is used as the endpoint to evaluate patient stratification strategy in UKB PPP OA patients as shown in FIG. 1 and FIG. 2.

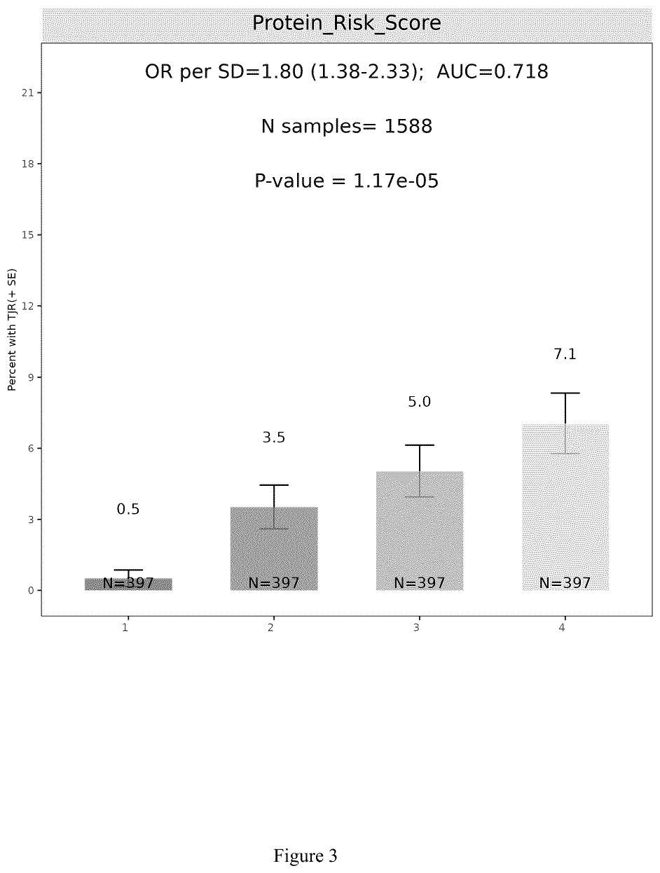

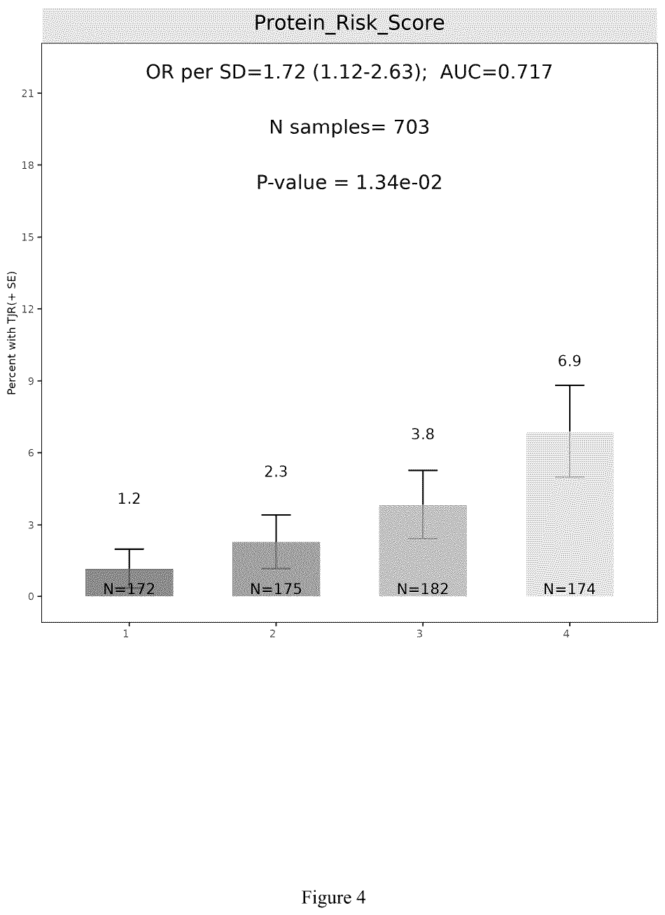

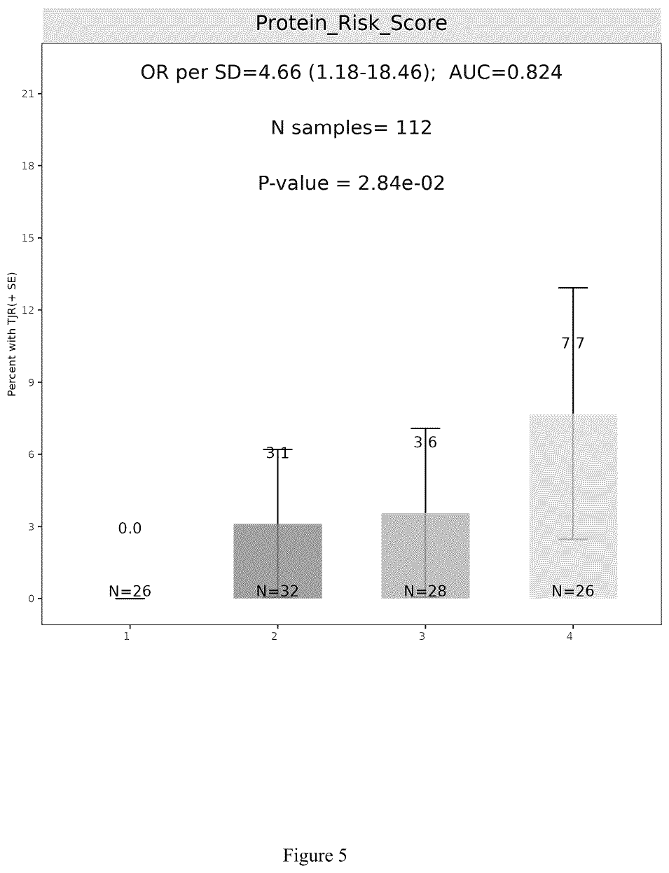

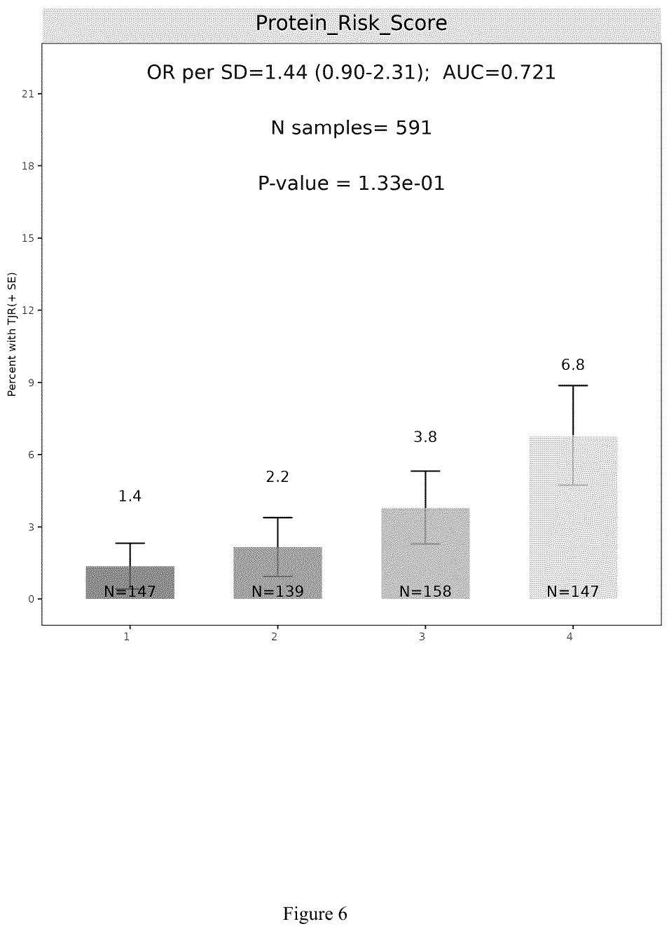

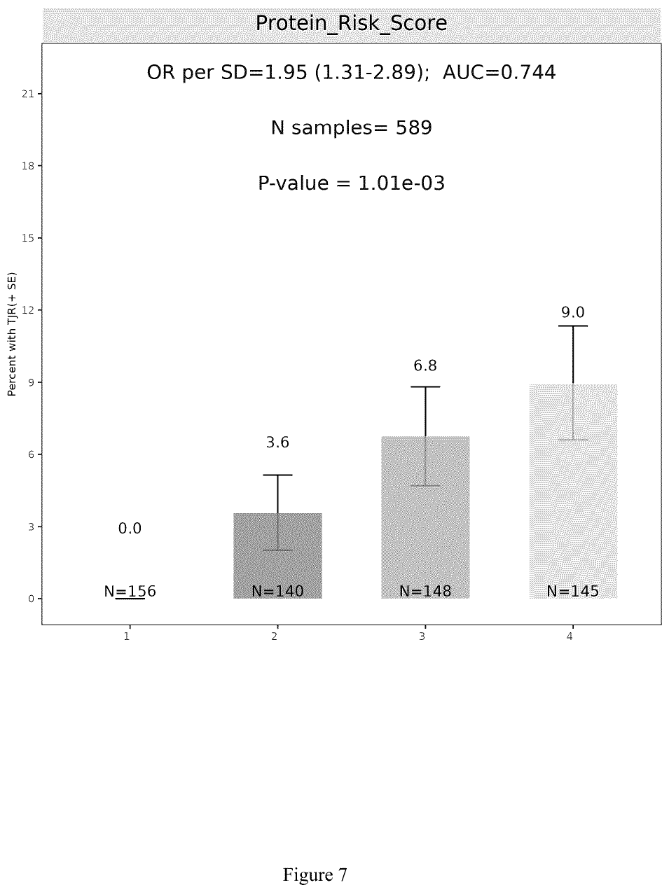

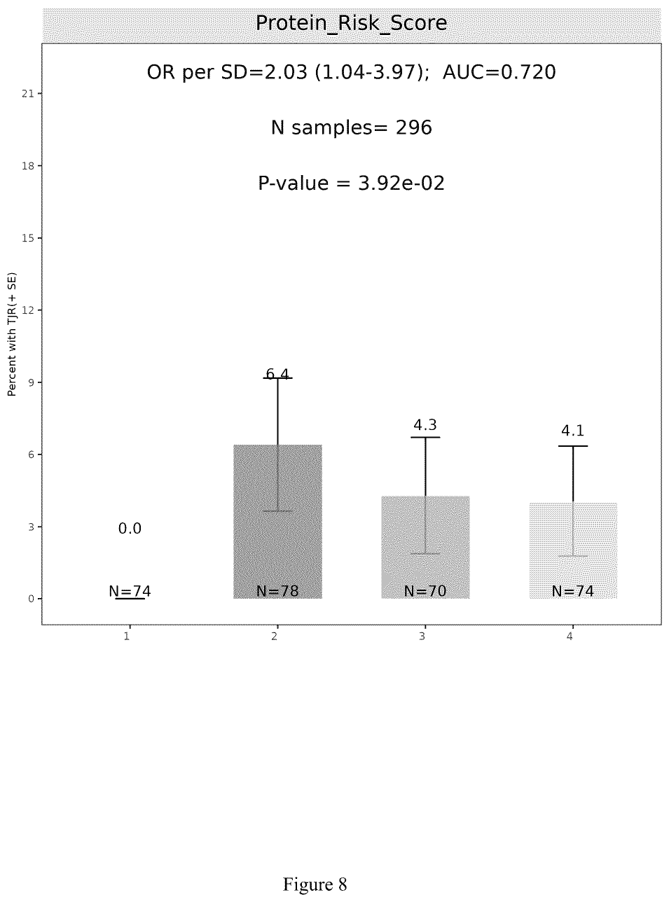

Finally, the total joint replacement is also used as the endpoint to evaluate OA proteomic score-based patient stratification in R475-OA-1688 clinical trial as shown in FIGS. 3-8. Patients in the 1^{st} to 3^{rd} OA proteomic score quartiles under fasinumab 1q4w treatment show a similar (or lower) total joint replacement percentage compared with patients in 1st to 3rd OA proteomic score quartiles receiving either NSAIDs or placebo treatment.

TABLE 3
Percentage of TJR in R475-OA-1688 by treatment arm

| Treatment Arm | 1^{st}-3^{rd} PRS Quartile | 4^{th} PRS Quartile |
|---|---|---|
| All Patients | 3.0% | 7.1% |
| Fasinumab all doses | 2.5% | 6.9% |
| Fasinumab high doses | 2.3% | 7.7% |
| Fasinumab 1q4w | 2.5% | 6.8% |
| NSAIDS | 3.4% | 9.0% |
| Placebo | 3.6% | 4.1% |

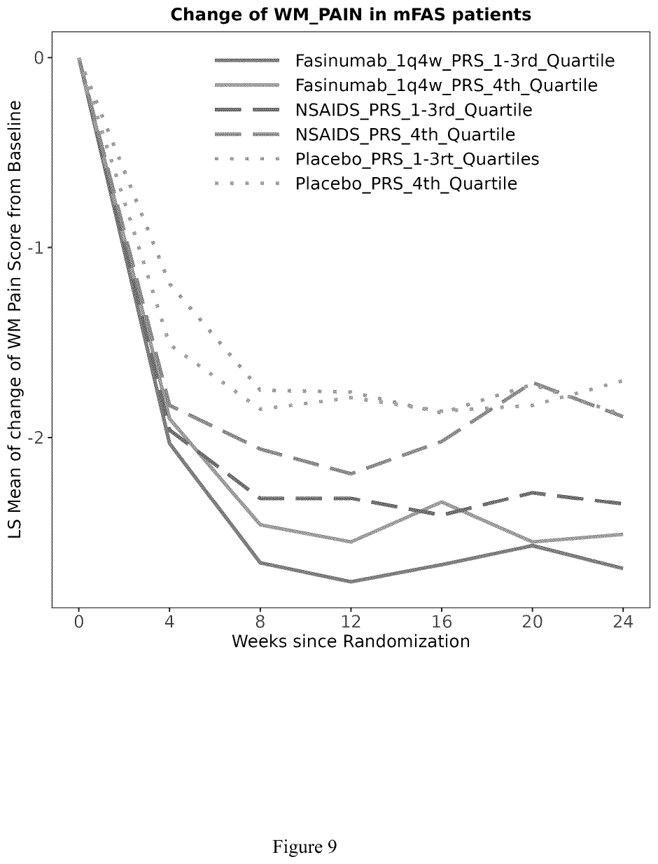

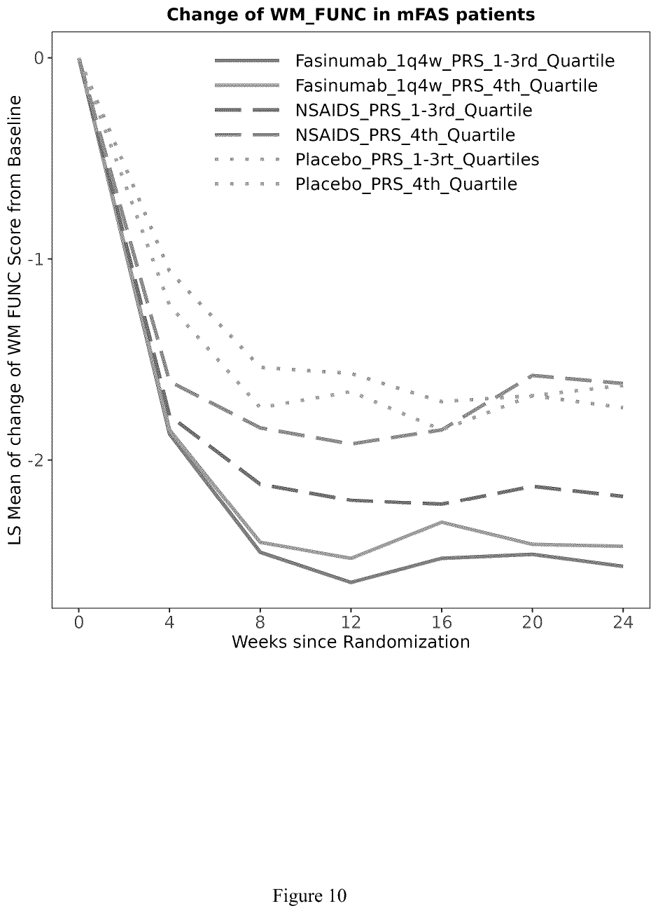

Besides identifying OA patients with better lower joint replacement progression rates, patients in 1^{st} to 3^{rd} OA proteomic score quartiles under fasinumab 1q4w and NSAIDs also demonstrated better efficacy profile (both WOMAC pain and WOMAC functional scores) as shown in FIG. 9 and FIG. 10. Also shown in FIG. 9 and FIG. 10, for NSAID treated patients with the highest risk quartile (4^{th} quartile) showed diminishing treatment response over time, to the extent that by week 24 NSAID high risk patients (4^{th} quartile) demonstrated response rates similar to placebo treated patients. Furthermore, Table 4 shows that fasinumab 1q4w maintains efficacy superiority compared with either NSAIDs or placebo in patients in 1^{st} to 3^{rd} OA proteomic score quartiles.

TABLE 4
WM Pain and Functional Score improvement at Week 24 in
OA PRS low risk group (1-3rd OA PRS Quartiles)

| | WM | WM |
|---|---|---|
| | Pain Score | Functional Score |
| Compare Group | (p-values) | (p-values) |
| Fasinumab 1q4w vs NSAIDs | 0.053 | 0.036 |
| Fasinumab 1q4w vs Celecoxib | 0.178 | 0.176 |
| Fasinumab 1q4w vs Diclofenac | 0.063 | 0.034 |
| Fasinumab 1q4w vs Placebo | 1.1e−4 | 1.3e−4 |

Table 5 shows that even in the OA proteomic score high risk group, Fasinumab 1q4w has efficacy benefits compared with both NSAIDs or placebo treated patients. NSAIDs treated patients in OA proteomic score high risk group do not demonstrate improved treatment response compared with placebo treated patients.

TABLE 5
WM Pain and Functional Score improvement at Week 24
in OA PRS high risk group (4th OA PRS Quartiles)

| | WM | WM |
|---|---|---|
| | Pain Score | Functional Score |
| | (p-values) | (p-values) |
| Fasinumab 1q4w vs NSAIDs | 0.028 | 0.003 |
| Fasinumab 1q4w vs Celecoxib | 0.019 | 0.049 |
| Fasinumab 1q4w vs Diclofenac | 0.022 | 0.005 |
| Fasinumab 1q4w vs Placebo | 0.013 | 0.017 |
| NSAIDs vs Placebo | 0.59 | 0.98 |
| Celecoxib vs Placebo | 0.99 | 0.70 |
| Diclofenac vs Placebo | 0.33 | 0.70 |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of determining the risk of a subject for developing osteoarthritis (OA), the method comprising:
determining or having determined the subject's osteoarthritis proteomic risk score, wherein the osteoarthritis proteomic risk score comprises a weighted aggregate of the expression level or circulating level of one or more proteins associated with OA;
wherein:
when the subject's osteoarthritis proteomic risk score is greater than or equal to a threshold osteoarthritis proteomic risk score, the subject has an increased risk of developing OA; and
when the subject's osteoarthritis proteomic risk score is less than a threshold osteoarthritis proteomic risk score, the subject has a decreased risk of developing OA; and
administering a Nerve Growth Factor (NGF) antagonist and/or a therapeutic agent that treats OA to the subject that has an osteoarthritis proteomic risk score that is less than or equal to a threshold osteoarthritis proteomic risk score.

2. The method of claim 1, wherein the one or more proteins associated with OA comprises Collagen alpha-1(IX) chain (COL9A1), Cartilage acidic protein 1 (CRTAC1), Scrapie-responsive protein 1 (SCRG1), Aggrecan core protein (ACAN), CCN family member 3 (CCN3), Chromogranin-A (CHGA), Kallikrein-4 (KLK4), RNA binding protein fox-1 homolog 3 (RBFOX3), Decorin (DCN), Matrilin-3 (MATN3), Cartilage oligomeric matrix protein (COMP), CUB domain-containing protein 1 (CDCP1), Protein BRICK1 (BRK1), Gastrin (GAST), Collagen alpha-3(VI) chain (COL6A3), Protein AMBP (AMBP), V-set and immunoglobulin domain-containing protein 2 (VSIG2), Fibroblast growth factor-binding protein 2 (FGFBP2), Annexin A10 (ANXA10), Chymotrypsin-like elastase family member 2A (CELA2A), T-cell immunoglobulin and mucin domain-containing protein 4 (TIMD4), Osteopontin (SPP1), Protein delta homolog 1 (DLK1), Pepsin A-4 (PGA4), Elastin (ELN), Insulin-like growth factor-binding protein 4 (IGFBP4), Fatty acid-binding protein, heart (FABP3), Triggering receptor expressed on myeloid cells 2 (TREM2), Transmembrane glycoprotein NMB (GPNMB), Cytokine-like protein 1 (CYTL1), Cytoskeleton-associated protein 4 (CKAP4), CD276 antigen (CD276), Ephrin type-A receptor 2 (EPHA2), Heat shock protein beta-6 (HSPB6), Adhesion G-protein coupled receptor G2 (ADGRG2), Myomesin-3 (MYOM3), Leukotriene A-4 hydrolase (LTA4H), Myosin-binding protein C, slow-type (MYBPC1), Collagenase 3 (MMP13), Neuronal calcium sensor 1 (NCS1), Coiled-coil-helix-coiled-coil-helix domain-containing protein 10, mitochondrial (CHCHD10), Myosin light chain 3 (MYL3), Macrophage metalloelastase (MMP12), Hepatitis A virus cellular receptor 2 (HAVCR2), Lamin-B2 (LMNB2), Trefoil factor 1 (TFF1), Tumor necrosis factor receptor superfamily member 27 (EDA2R), Neurocan core protein (NCAN), Asialoglycoprotein receptor 1 (ASGR1), Angiotensin-converting enzyme 2 (ACE2), C4b-binding protein beta chain (C4BPB), Protein disulfide isomerase CRELD1 (CRELD1), Chitinase-3-like protein 1 (CHI3L1), Protein kinase C-binding protein NELL2 (NELL2), Contactin-1 (CNTN1), Branched-chain-amino-acid aminotransferase, cytosolic (BCAT1), Transcriptional coactivator YAP1 (YAP1), CD302 antigen (CD302), Lactoperoxidase (LPO), Natural cytotoxicity triggering receptor 3 ligand 1 (NCR3LG1), MANSC domain-containing protein 4 (MANSC4), Heparan-sulfate 6-O-sulfotransferase 2 (HS6ST2), Uromodulin (UMOD), Amphiregulin (AREG), Disintegrin and metalloproteinase domain-containing protein 12 (ADAM12), Insulin-like growth factor-binding protein 1 (IGFBP1), Secretin (SCT), Collectin-12 (COLEC12), Prolargin (PRELP), Alpha-actinin-2 (ACTN2), Occludin (OCLN), Galectin-3 (LGALS3), Cathepsin L1 (CTSL), Dipeptidyl aminopeptidase-like protein 6 (DPP6), Transthyretin (TTR), Gastric triacylglycerol lipase (LIPF), Thyrotropin subunit beta (TSHB), Urokinase plasminogen activator surface receptor (PLAUR), Contactin-2 (CNTN2), Persephin (PSPN), Arginase-1 (ARG1), Uteroglobin (SCGB1A1), BPI fold-containing family A member 2 (BPIFA2), Follistatin (FST), Insulin-like growth factor-binding protein 2 (IGFBP2), Anterior gradient protein 2 homolog (AGR2), Trefoil factor 2 (TFF2), Neuronal cell adhesion molecule (NRCAM), Sushi domain-containing protein 5 (SUSD5), Leukocyte immunoglobulin-like receptor subfamily B member 4 (LILRB4), Protein turtle homolog A (IGSF9), Tenascin (TNC), Pyruvate kinase PKLR (PKLR), Thiamin pyrophosphokinase 1 (TPK1), UL16-binding protein 2 (ULBP2), Hypoxia up-regulated protein 1 (HYOU1), Inactive dipeptidyl peptidase 10 (DPP10), Flavin reductase (NADPH) (BLVRB), Carbonic anhydrase 1 (CA1), Ecto-ADP-ribosyltransferase 3 (ART3), Protein kinase C-binding protein NELL1 (NELL1), Macrophage scavenger receptor types I and II (MSR1), Tumor necrosis factor receptor superfamily member 10A (TNFRSF10A), Hepatitis A virus cellular receptor 1 (HAVCR1), Peroxiredoxin-2 (PRDX2), Chondroitin sulfate proteoglycan 4 (CSPG4), Growth/differentiation factor 15 (GDF15), Leucine-rich repeat neuronal protein 1 (LRRN1), Tumor necrosis factor receptor superfamily member 12A (TNFRSF12A), Cadherin-related family member 2 (CDHR2), Calcineurin subunit B type 1 (PPP3R1), Tumor necrosis factor receptor superfamily member 9 (TNFRSF9), Brevican core protein (BCAN), Cerebellin-4 (CBLN4), Creatine kinase B-type (CKB), Disintegrin and metalloproteinase domain-containing protein 9 (ADAM9), Nectin-2 (NECTIN2), Down syndrome cell adhesion molecule (DSCAM), Uncharacterized protein C9orf40 (C9orf40), Protein DDI1 homolog 2 (DDI2), Stromelysin-1 (MMP3), Adiponectin (ADIPOQ), CMP-N-acetylneuraminate-beta-galactosamide-alpha-2,3-sialyltransferase 1 (ST3GAL1), Layilin (LAYN), Porphobilinogen deaminase (HMBS), Tumor necrosis factor receptor superfamily member 3 (LTBR), Lysosomal Pro-X carboxypeptidase (PRCP), Centromere protein F (CENPF), Chymotrypsin-like elastase family member 3A (CELA3A), Carbonic anhydrase 12 (CA12), SLIT and NTRK-like protein 1 (SLITRK1), Myomesin-2 (MYOM2), Receptor tyrosine-protein kinase erbB-4 (ERBB4), Lysosome membrane protein 2 (SCARB2), Collagen alpha-1(XVIII) chain (COL18A1), Low-density lipoprotein receptor-related protein 11 (LRP11), Thrombospondin-4 (THBS4), Pleiotrophin (PTN), Phosphoethanolamine/phosphocholine phosphatase (PHOSPHO1), Asialoglycoprotein receptor 2 (ASGR2), Retinoic acid receptor responder protein 2 (RARRES2), C-C motif chemokine 7 (CCL7), Stanniocalcin-1 (STC1), Osteocalcin (BGLAP), Interleukin-15 (IL15), Protein-glutamine gamma-glutamyltransferase 2 (TGM2), Latent-transforming growth factor beta-binding protein 2 (LTBP2), Gamma-interferon-inducible lysosomal thiol reductase (IFI30), Serine protease inhibitor Kazal-type 5 (SPINK5), WAP, Kazal, immunoglobulin, Kunitz and NTR domain-containing protein 1 (WFIKKN1), Ephrin type-B receptor 4 (EPHB4), Dystrobrevin beta (DTNB), Neutrophil cytosol factor 2 (NCF2), Mimecan (OGN), Alpha-hemoglobin-stabilizing protein (AHSP), Tumor necrosis factor receptor superfamily member 14 (TNFRSF14), Tectonic-3 (TCTN3), AMP deaminase 3 (AMPD3), Tumor necrosis factor receptor superfamily member 11B (TNFRSF11B), Protein enabled homolog (ENAH), Galectin-1 (LGALS1), Hydroxyacylglutathione hydrolase, mitochondrial (HAGH), Fetuin-B (FETUB), Fibronectin type III domain-containing protein 1 (FNDC1), C-type lectin domain family 5 member A (CLEC5A), A disintegrin and metalloproteinase with thrombospondin motifs 13 (ADAMTS13), Cysteine and glycine-rich protein 3 (CSRP3), Keratin, type I cytoskeletal 19 (KRT19), Cytokine receptor-like factor 1 (CRLF1), BAG family molecular chaperone regulator 3 (BAG3), Sclerostin (SOST), Fc receptor-like protein 5 (FCRL5), Acetylcholinesterase (ACHE), Actin, aortic smooth muscle (ACTA2), Coiled-coil domain-containing protein 80 (CCDC80), Odontogenic ameloblast-associated protein (ODAM), Cystatin-B (CSTB), Seizure 6-like protein (SEZ6L), Stanniocalcin-2 (STC2), Myosin light chain 4 (MYL4), Retinal dehydrogenase 1 (ALDH1A1), Matrix remodeling-associated protein 8 (MXRA8), Macrophage-capping protein (CAPG), Band 3 anion transport protein (SLC4A1), Chymotrypsinogen B (CTRB1), Trefoil factor 3 (TFF3), C-type natriuretic peptide (NPPC), Cystatin-D (CST5), Prolow-density lipoprotein receptor-related protein 1 (LRP1), C-C motif chemokine 5 (CCL5), Carbonic anhydrase 2 (CA2), Pregnancy-specific beta-1-glycoprotein 1 (PSG1), Gastrotropin (FABP6), Ephrin-A1 (EFNA1), Thrombomodulin (THBD), DnaJ homolog subfamily A member 4 (DNAJA4), Interleukin-4 receptor subunit alpha (IL4R), Glycerophosphocholine cholinephosphodiesterase ENPP6 (ENPP6), or Parathyroid hormone (PTH), or any combination thereof.

3. The method of claim 1, wherein the one or more proteins associated with OA comprises COL9A1, CRTAC1, CNTN1, GAST, CYTL1, KLK4, DLK1, CDCP1, DPP10, CELA2A, RBFOX3, CRELD1, NELL2, PGA4, ARG1, ART3, TIMD4, FGFBP2, BPIFA2, PTH, or BRK1, or any combination thereof.

4-6. (canceled)

7. The method of claim 1, wherein the osteoarthritis proteomic risk score is determined from a biological sample obtained from the subject, wherein the biological sample comprises blood, serum, plasma, semen, saliva, urine, feces, hair, teeth, bone, tissue, a swab from a cheek, or a cell.

8. (canceled)

9. The method of claim 1, wherein the NGF antagonist is an antibody, a bispecific antibody, a polypeptide, an antisense nucleic acid molecule, an siRNA molecule, or a small molecule.

10. The method of claim 9, wherein the antibody is fasinumab, tanezumab, MEDI7352, or fulranumab.

11. (canceled)

12. The method of claim 9, wherein the small molecule is K252a, ALE-0540, PQC-083, PD-90780, LM11A-31 dihydrochloride, Y1036, GZ389988A, or Ro 08-2750.

13. A method of determining the risk of a subject progressing to total joint replacement (TJR), the method comprising:
determining or having determined the subject's osteoarthritis proteomic risk score, wherein the osteoarthritis proteomic risk score comprises a weighted aggregate of the expression level or circulating level of one or more proteins associated with OA;
wherein:
when the subject's osteoarthritis proteomic risk score is greater than or equal to a threshold osteoarthritis proteomic risk score, the subject has an increased risk of progressing to TJR; and
when the subject's osteoarthritis proteomic risk score is less than a threshold osteoarthritis proteomic risk score, the subject has a decreased risk of progressing to TJR; and
administering a Nerve Growth Factor (NGF) antagonist and/or a therapeutic agent that treats OA to the subject that has an osteoarthritis proteomic risk score that is less than or equal to a threshold osteoarthritis proteomic risk score.

14. The method of claim 13, wherein the one or more proteins associated with OA comprises Collagen alpha-1(IX) chain (COL9A1), Cartilage acidic protein 1 (CRTAC1), Scrapie-responsive protein 1 (SCRG1), Aggrecan core protein (ACAN), CCN family member 3 (CCN3), Chromogranin-A (CHGA), Kallikrein-4 (KLK4), RNA binding protein fox-1 homolog 3 (RBFOX3), Decorin (DCN), Matrilin-3 (MATN3), Cartilage oligomeric matrix protein (COMP), CUB domain-containing protein 1 (CDCP1), Protein BRICK1 (BRK1), Gastrin (GAST), Collagen alpha-3(VI) chain (COL6A3), Protein AMBP (AMBP), V-set and immunoglobulin domain-containing protein 2 (VSIG2), Fibroblast growth factor-binding protein 2 (FGFBP2), Annexin A10 (ANXA10), Chymotrypsin-like elastase family member 2A (CELA2A), T-cell immunoglobulin and mucin domain-containing protein 4 (TIMD4), Osteopontin (SPP1), Protein delta homolog 1 (DLK1), Pepsin A-4 (PGA4), Elastin (ELN), Insulin-like growth factor-binding protein 4 (IGFBP4), Fatty acid-binding protein, heart (FABP3), Triggering receptor expressed on myeloid cells 2 (TREM2), Transmembrane glycoprotein NMB (GPNMB), Cytokine-like protein 1 (CYTL1), Cytoskeleton-associated protein 4 (CKAP4), CD276 antigen (CD276), Ephrin type-A receptor 2 (EPHA2), Heat shock protein beta-6 (HSPB6), Adhesion G-protein coupled receptor G2 (ADGRG2), Myomesin-3 (MYOM3), Leukotriene A-4 hydrolase (LTA4H), Myosin-binding protein C, slow-type (MYBPC1), Collagenase 3 (MMP13), Neuronal calcium sensor 1 (NCS1), Coiled-coil-helix-coiled-coil-helix domain-containing protein 10, mitochondrial (CHCHD10), Myosin light chain 3 (MYL3), Macrophage metalloelastase (MMP12), Hepatitis A virus cellular receptor 2 (HAVCR2), Lamin-B2 (LMNB2), Trefoil factor 1 (TFF1), Tumor necrosis factor receptor superfamily member 27 (EDA2R), Neurocan core protein (NCAN), Asialoglycoprotein receptor 1 (ASGR1), Angiotensin-converting enzyme 2 (ACE2), C4b-binding protein beta chain (C4BPB), Protein disulfide isomerase CRELD1 (CRELD1), Chitinase-3-like protein 1 (CHI3L1), Protein kinase C-binding protein NELL2 (NELL2), Contactin-1 (CNTN1), Branched-chain-amino-acid aminotransferase, cytosolic (BCAT1), Transcriptional coactivator YAP1 (YAP1), CD302 antigen (CD302), Lactoperoxidase (LPO), Natural cytotoxicity triggering receptor 3 ligand 1 (NCR3LG1), MANSC domain-containing protein 4 (MANSC4), Heparan-sulfate 6-O-sulfotransferase 2 (HS6ST2), Uromodulin (UMOD), Amphiregulin (AREG), Disintegrin and metalloproteinase domain-containing protein 12 (ADAM12), Insulin-like growth factor-binding protein 1 (IGFBP1), Secretin (SCT), Collectin-12 (COLEC12), Prolargin (PRELP), Alpha-actinin-2 (ACTN2), Occludin (OCLN), Galectin-3 (LGALS3), Cathepsin L1 (CTSL), Dipeptidyl aminopeptidase-like protein 6 (DPP6), Transthyretin (TTR), Gastric triacylglycerol lipase (LIPF), Thyrotropin subunit beta (TSHB), Urokinase plasminogen activator surface receptor (PLAUR), Contactin-2 (CNTN2), Persephin (PSPN), Arginase-1 (ARG1), Uteroglobin (SCGB1A1), BPI fold-containing family A member 2 (BPIFA2), Follistatin (FST), Insulin-like growth factor-binding protein 2 (IGFBP2), Anterior gradient protein 2 homolog (AGR2), Trefoil factor 2 (TFF2), Neuronal cell adhesion molecule (NRCAM), Sushi domain-containing protein 5 (SUSD5), Leukocyte immunoglobulin-like receptor subfamily B member 4 (LILRB4), Protein turtle homolog A (IGSF9), Tenascin (TNC), Pyruvate kinase PKLR (PKLR), Thiamin pyrophosphokinase 1 (TPK1), UL16-binding protein 2 (ULBP2), Hypoxia up-regulated protein 1 (HYOU1), Inactive dipeptidyl peptidase 10 (DPP10), Flavin reductase (NADPH) (BLVRB), Carbonic anhydrase 1 (CA1), Ecto-ADP-ribosyltransferase 3 (ART3), Protein kinase C-binding protein NELL1 (NELL1), Macrophage scavenger receptor types I and II (MSR1), Tumor necrosis factor receptor superfamily member 10A (TNFRSF10A), Hepatitis A virus cellular receptor 1 (HAVCR1), Peroxiredoxin-2 (PRDX2), Chondroitin sulfate proteoglycan 4 (CSPG4), Growth/differentiation factor 15 (GDF15), Leucine-rich repeat neuronal protein 1 (LRRN1), Tumor necrosis factor receptor superfamily member 12A (TNFRSF12A), Cadherin-related family member 2 (CDHR2), Calcineurin subunit B type 1 (PPP3R1), Tumor necrosis factor receptor superfamily member 9 (TNFRSF9), Brevican core protein (BCAN), Cerebellin-4 (CBLN4), Creatine kinase B-type (CKB), Disintegrin and metalloproteinase domain-containing protein 9 (ADAM9), Nectin-2 (NECTIN2), Down syndrome cell adhesion molecule (DSCAM), Uncharacterized protein C9orf40 (C9orf40), Protein DDI1 homolog 2 (DDI2), Stromelysin-1 (MMP3), Adiponectin (ADIPOQ), CMP-N-acetylneuraminate-beta-galactosamide-alpha-2,3-sialyltransferase 1 (ST3GAL1), Layilin (LAYN), Porphobilinogen deaminase (HMBS), Tumor necrosis factor receptor superfamily member 3 (LTBR), Lysosomal Pro-X carboxypeptidase (PRCP), Centromere protein F (CENPF), Chymotrypsin-like elastase family member 3A (CELA3A), Carbonic anhydrase 12 (CA12), SLIT and NTRK-like protein 1 (SLITRK1), Myomesin-2 (MYOM2), Receptor tyrosine-protein kinase erbB-4 (ERBB4), Lysosome membrane protein 2 (SCARB2), Collagen alpha-1(XVIII) chain (COL18A1), Low-density lipoprotein receptor-related protein 11 (LRP11), Thrombospondin-4 (THBS4), Pleiotrophin (PTN), Phosphoethanolamine/phosphocholine phosphatase (PHOSPHO1), Asialoglycoprotein receptor 2 (ASGR2), Retinoic acid receptor responder protein 2 (RARRES2), C-C motif chemokine 7 (CCL7), Stanniocalcin-1 (STC1), Osteocalcin (BGLAP), Interleukin-15 (IL15), Protein-glutamine gamma-glutamyltransferase 2 (TGM2), Latent-transforming growth factor beta-binding protein 2 (LTBP2), Gamma-interferon-inducible lysosomal thiol reductase (IFI30), Serine protease inhibitor Kazal-type 5 (SPINK5), WAP, Kazal, immunoglobulin, Kunitz and NTR domain-containing protein 1 (WFIKKN1), Ephrin type-B receptor 4 (EPHB4), Dystrobrevin beta (DTNB), Neutrophil cytosol factor 2 (NCF2), Mimecan (OGN), Alpha-hemoglobin-stabilizing protein (AHSP), Tumor necrosis factor receptor superfamily member 14 (TNFRSF14), Tectonic-3 (TCTN3), AMP deaminase 3 (AMPD3), Tumor necrosis factor receptor superfamily member 11B (TNFRSF11B), Protein enabled homolog (ENAH), Galectin-1 (LGALS1), Hydroxyacylglutathione hydrolase, mitochondrial (HAGH), Fetuin-B (FETUB), Fibronectin type III domain-containing protein 1 (FNDC1), C-type lectin domain family 5 member A (CLEC5A), A disintegrin and metalloproteinase with thrombospondin motifs 13 (ADAMTS13), Cysteine and glycine-rich protein 3 (CSRP3), Keratin, type I cytoskeletal 19 (KRT19), Cytokine receptor-like factor 1 (CRLF1), BAG family molecular chaperone regulator 3 (BAG3), Sclerostin (SOST), Fc receptor-like protein 5 (FCRL5), Acetylcholinesterase (ACHE), Actin, aortic smooth muscle (ACTA2), Coiled-coil domain-containing protein 80 (CCDC80), Odontogenic ameloblast-associated protein (ODAM), Cystatin-B (CSTB), Seizure 6-like protein (SEZ6L), Stanniocalcin-2 (STC2), Myosin light chain 4 (MYL4), Retinal dehydrogenase 1 (ALDH1A1), Matrix remodeling-associated protein 8 (MXRA8), Macrophage-capping protein (CAPG), Band 3 anion transport protein (SLC4A1), Chymotrypsinogen B (CTRB1), Trefoil factor 3 (TFF3), C-type natriuretic peptide (NPPC), Cystatin-D (CST5), Prolow-density lipoprotein receptor-related protein 1 (LRP1), C-C motif chemokine 5 (CCL5), Carbonic anhydrase 2 (CA2), Pregnancy-specific beta-1-glycoprotein 1 (PSG1), Gastrotropin (FABP6), Ephrin-A1 (EFNA1), Thrombomodulin (THBD), DnaJ homolog subfamily A member 4 (DNAJA4), Interleukin-4 receptor subunit alpha (IL4R), Glycerophosphocholine cholinephosphodiesterase ENPP6 (ENPP6), or Parathyroid hormone (PTH), or any combination thereof.

15. The method of claim 13, wherein the one or more proteins associated with OA comprises COL9A1, CRTAC1, CNTN1, GAST, CYTL1, KLK4, DLK1, CDCP1, DPP10, CELA2A, RBFOX3, CRELD1, NELL2, PGA4, ARG1, ART3, TIMD4, FGFBP2, BPIFA2, PTH, or BRK1, or any combination thereof.

16-18. (canceled)

19. The method of claim 13, wherein the osteoarthritis proteomic risk score is determined from a biological sample obtained from the subject, wherein the biological sample comprises blood, serum, plasma, semen, saliva, urine, feces, hair, teeth, bone, tissue, a swab from a cheek, or a cell.

20. (canceled)

21. The method of claim 13, wherein the NGF antagonist is an antibody, bispecific antibody, a polypeptide, an antisense nucleic acid molecule, an siRNA molecule, or a small molecule.

22. The method of claim 21, wherein the antibody is fasinumab, tanezumab, MEDI7352, or fulranumab.

23. (canceled)

24. The method of claim 21, wherein the small molecule is K252a, ALE-0540, PQC-083, PD-90780, LM11A-31 dihydrochloride, Y1036, GZ389988A, or Ro 08-2750.

25-43. (canceled)

44. A method of determining the treatment response of a subject (pain and functional improvement), the method comprising:
determining or having determined the subject's osteoarthritis proteomic risk score, wherein the osteoarthritis proteomic risk score comprises a weighted aggregate of the expression level or circulating level of one or more proteins associated with OA;
wherein:
when the subject's osteoarthritis proteomic risk score is greater than or equal to a threshold osteoarthritis proteomic risk score, the subject has less treatment response; and
when the subject's osteoarthritis proteomic risk score is less than a threshold osteoarthritis proteomic risk score, the subject has an increase in treatment response; and
administering a Nerve Growth Factor (NGF) antagonist and/or a therapeutic agent that treats OA to the subject that has an osteoarthritis proteomic risk score that is less than or equal to a threshold osteoarthritis proteomic risk score; or
administering a Non-Steroidal Anti-inflammatory Drug (NSAID) and/or a therapeutic agent that treats OA to the subject that has an osteoarthritis proteomic risk score that is less than or equal to a threshold osteoarthritis proteomic risk score; or
administering an opioid and/or a therapeutic agent that treats OA to the subject that has an osteoarthritis proteomic risk score that is less than or equal to a threshold osteoarthritis proteomic risk score; or
administering a NaV1.8 or a NaV1.7 inhibitor and/or a therapeutic agent that treats OA to the subject that has an osteoarthritis proteomic risk score that is less than or equal to a threshold osteoarthritis proteomic risk score.

45. The method of claim 44, wherein the one or more proteins associated with OA comprises Collagen alpha-1(IX) chain (COL9A1), Cartilage acidic protein 1 (CRTAC1), Scrapie-responsive protein 1 (SCRG1), Aggrecan core protein (ACAN), CCN family member 3 (CCN3), Chromogranin-A (CHGA), Kallikrein-4 (KLK4), RNA binding protein fox-1 homolog 3 (RBFOX3), Decorin (DCN), Matrilin-3 (MATN3), Cartilage oligomeric matrix protein (COMP), CUB domain-containing protein 1 (CDCP1), Protein BRICK1 (BRK1), Gastrin (GAST), Collagen alpha-3(VI) chain (COL6A3), Protein AMBP (AMBP), V-set and immunoglobulin domain-containing protein 2 (VSIG2), Fibroblast growth factor-binding protein 2 (FGFBP2), Annexin A10 (ANXA10), Chymotrypsin-like elastase family member 2A (CELA2A), T-cell immunoglobulin and mucin domain-containing protein 4 (TIMD4), Osteopontin (SPP1), Protein delta homolog 1 (DLK1), Pepsin A-4 (PGA4), Elastin (ELN), Insulin-like growth factor-binding protein 4 (IGFBP4), Fatty acid-binding protein, heart (FABP3), Triggering receptor expressed on myeloid cells 2 (TREM2), Transmembrane glycoprotein NMB (GPNMB), Cytokine-like protein 1 (CYTL1), Cytoskeleton-associated protein 4 (CKAP4), CD276 antigen (CD276), Ephrin type-A receptor 2 (EPHA2), Heat shock protein beta-6 (HSPB6), Adhesion G-protein coupled receptor G2 (ADGRG2), Myomesin-3 (MYOM3), Leukotriene A-4 hydrolase (LTA4H), Myosin-binding protein C, slow-type (MYBPC1), Collagenase 3 (MMP13), Neuronal calcium sensor 1 (NCS1), Coiled-coil-helix-coiled-coil-helix domain-containing protein 10, mitochondrial (CHCHD10), Myosin light chain 3 (MYL3), Macrophage metalloelastase (MMP12), Hepatitis A virus cellular receptor 2 (HAVCR2), Lamin-B2 (LMNB2), Trefoil factor 1 (TFF1), Tumor necrosis factor receptor superfamily member 27 (EDA2R), Neurocan core protein (NCAN), Asialoglycoprotein receptor 1 (ASGR1), Angiotensin-converting enzyme 2 (ACE2), C4b-binding protein beta chain (C4BPB), Protein disulfide isomerase CRELD1 (CRELD1), Chitinase-3-like protein 1 (CHI3L1), Protein kinase C-binding protein NELL2 (NELL2), Contactin-1 (CNTN1), Branched-chain-amino-acid aminotransferase, cytosolic (BCAT1), Transcriptional coactivator YAP1 (YAP1), CD302 antigen (CD302), Lactoperoxidase (LPO), Natural cytotoxicity triggering receptor 3 ligand 1 (NCR3LG1), MANSC domain-containing protein 4 (MANSC4), Heparan-sulfate 6-O-sulfotransferase 2 (HS6ST2), Uromodulin (UMOD), Amphiregulin (AREG), Disintegrin and metalloproteinase domain-containing protein 12 (ADAM12), Insulin-like growth factor-binding protein 1 (IGFBP1), Secretin (SCT), Collectin-12 (COLEC12), Prolargin (PRELP), Alpha-actinin-2 (ACTN2), Occludin (OCLN), Galectin-3 (LGALS3), Cathepsin L1 (CTSL), Dipeptidyl aminopeptidase-like protein 6 (DPP6), Transthyretin (TTR), Gastric triacylglycerol lipase (LIPF), Thyrotropin subunit beta (TSHB), Urokinase plasminogen activator surface receptor (PLAUR), Contactin-2 (CNTN2), Persephin (PSPN), Arginase-1 (ARG1), Uteroglobin (SCGB1A1), BPI fold-containing family A member 2 (BPIFA2), Follistatin (FST), Insulin-like growth factor-binding protein 2 (IGFBP2), Anterior gradient protein 2 homolog (AGR2), Trefoil factor 2 (TFF2), Neuronal cell adhesion molecule (NRCAM), Sushi domain-containing protein 5 (SUSD5), Leukocyte immunoglobulin-like receptor subfamily B member 4 (LILRB4), Protein turtle homolog A (IGSF9), Tenascin (TNC), Pyruvate kinase PKLR (PKLR), Thiamin pyrophosphokinase 1 (TPK1), UL16-binding protein 2 (ULBP2), Hypoxia up-regulated protein 1 (HYOU1), Inactive dipeptidyl peptidase 10 (DPP10), Flavin reductase (NADPH) (BLVRB), Carbonic anhydrase 1 (CA1), Ecto-ADP-ribosyltransferase 3 (ART3), Protein kinase C-binding protein NELL1 (NELL1), Macrophage scavenger receptor types I and II (MSR1), Tumor necrosis factor receptor superfamily member 10A (TNFRSF10A), Hepatitis A virus cellular receptor 1 (HAVCR1), Peroxiredoxin-2 (PRDX2), Chondroitin sulfate proteoglycan 4 (CSPG4), Growth/differentiation factor 15 (GDF15), Leucine-rich repeat neuronal protein 1 (LRRN1), Tumor necrosis factor receptor superfamily member 12A (TNFRSF12A), Cadherin-related family member 2 (CDHR2), Calcineurin subunit B type 1 (PPP3R1), Tumor necrosis factor receptor superfamily member 9 (TNFRSF9), Brevican core protein (BCAN), Cerebellin-4 (CBLN4), Creatine kinase B-type (CKB), Disintegrin and metalloproteinase domain-containing protein 9 (ADAM9), Nectin-2 (NECTIN2), Down syndrome cell adhesion molecule (DSCAM), Uncharacterized protein C9orf40 (C9orf40), Protein DDI1 homolog 2 (DDI2), Stromelysin-1 (MMP3), Adiponectin (ADIPOQ), CMP-N-acetylneuraminate-beta-galactosamide-alpha-2,3-sialyltransferase 1 (ST3GAL1), Layilin (LAYN), Porphobilinogen deaminase (HMBS), Tumor necrosis factor receptor superfamily member 3 (LTBR), Lysosomal Pro-X carboxypeptidase (PRCP), Centromere protein F (CENPF), Chymotrypsin-like elastase family member 3A (CELA3A), Carbonic anhydrase 12 (CA12), SLIT and NTRK-like protein 1 (SLITRK1), Myomesin-2 (MYOM2), Receptor tyrosine-protein kinase erbB-4 (ERBB4), Lysosome membrane protein 2 (SCARB2), Collagen alpha-1(XVIII) chain (COL18A1), Low-density lipoprotein receptor-related protein 11 (LRP11), Thrombospondin-4 (THBS4), Pleiotrophin (PTN), Phosphoethanolamine/phosphocholine phosphatase (PHOSPHO1), Asialoglycoprotein receptor 2 (ASGR2), Retinoic acid receptor responder protein 2 (RARRES2), C-C motif chemokine 7 (CCL7), Stanniocalcin-1 (STC1), Osteocalcin (BGLAP), Interleukin-15 (IL15), Protein-glutamine gamma-glutamyltransferase 2 (TGM2), Latent-transforming growth factor beta-binding protein 2 (LTBP2), Gamma-interferon-inducible lysosomal thiol reductase (IFI30), Serine protease inhibitor Kazal-type 5 (SPINK5), WAP, Kazal, immunoglobulin, Kunitz and NTR domain-containing protein 1 (WFIKKN1), Ephrin type-B receptor 4 (EPHB4), Dystrobrevin beta (DTNB), Neutrophil cytosol factor 2 (NCF2), Mimecan (OGN), Alpha-hemoglobin-stabilizing protein (AHSP), Tumor necrosis factor receptor superfamily member 14 (TNFRSF14), Tectonic-3 (TCTN3), AMP deaminase 3 (AMPD3), Tumor necrosis factor receptor superfamily member 11B (TNFRSF11B), Protein enabled homolog (ENAH), Galectin-1 (LGALS1), Hydroxyacylglutathione hydrolase, mitochondrial (HAGH), Fetuin-B (FETUB), Fibronectin type III domain-containing protein 1 (FNDC1), C-type lectin domain family 5 member A (CLEC5A), A disintegrin and metalloproteinase with thrombospondin motifs 13 (ADAMTS13), Cysteine and glycine-rich protein 3 (CSRP3), Keratin, type I cytoskeletal 19 (KRT19), Cytokine receptor-like factor 1 (CRLF1), BAG family molecular chaperone regulator 3 (BAG3), Sclerostin (SOST), Fc receptor-like protein 5 (FCRL5), Acetylcholinesterase (ACHE), Actin, aortic smooth muscle (ACTA2), Coiled-coil domain-containing protein 80 (CCDC80), Odontogenic ameloblast-associated protein (ODAM), Cystatin-B (CSTB), Seizure 6-like protein (SEZ6L), Stanniocalcin-2 (STC2), Myosin light chain 4 (MYL4), Retinal dehydrogenase 1 (ALDH1A1), Matrix remodeling-associated protein 8 (MXRA8), Macrophage-capping protein (CAPG), Band 3 anion transport protein (SLC4A1), Chymotrypsinogen B (CTRB1), Trefoil factor 3 (TFF3), C-type natriuretic peptide (NPPC), Cystatin-D (CST5), Prolow-density lipoprotein receptor-related protein 1 (LRP1), C-C motif chemokine 5 (CCL5), Carbonic anhydrase 2 (CA2), Pregnancy-specific beta-1-glycoprotein 1 (PSG1), Gastrotropin (FABP6), Ephrin-A1 (EFNA1), Thrombomodulin (THBD), DnaJ homolog subfamily A member 4 (DNAJA4), Interleukin-4 receptor subunit alpha (IL4R), Glycerophosphocholine cholinephosphodiesterase ENPP6 (ENPP6), or Parathyroid hormone (PTH), or any combination thereof.

46. The method of claim 44, wherein the one or more proteins associated with OA comprises COL9A1, CRTAC1, CNTN1, GAST, CYTL1, KLK4, DLK1, CDCP1, DPP10, CELA2A, RBFOX3, CRELD1, NELL2, PGA4, ARG1, ART3, TIMD4, FGFBP2, BPIFA2, PTH, or BRK1, or any combination thereof.

47-49. (canceled)

50. The method of claim 44, wherein the osteoarthritis proteomic risk score is determined from a biological sample obtained from the subject, wherein the biological sample comprises blood, serum, plasma, semen, saliva, urine, feces, hair, teeth, bone, tissue, a swab from a cheek, or a cell.

51. (canceled)

52. The method of claim 44, wherein the NGF antagonist comprises an antibody, bispecific antibody, a polypeptide, an antisense nucleic acid molecule, an siRNA molecule, or a small molecule.

53. The method of claim 52, wherein the antibody comprises fasinumab, tanezumab, MEDI7352, or fulranumab.

54. (canceled)

55. The method of claim 52, wherein the small molecule comprises K252a, ALE-0540, PQC-083, PD-90780, LM11A-31 dihydrochloride, Y1036, GZ389988A, or Ro 08-2750.

56. (canceled)

57. The method of claim 44, where the NSAID comprises ibuprofen, naproxen, diclofenac, etodolac, meloxicam, oxaprozin, celecoxib, piroxicam, indomethacin, or sulindac, or any combination thereof.

58. (canceled)

59. The method of claim 44, where the opioid comprises codeine, hydrocodone, oxycodone, or tramadol, or any combination thereof.

60. (canceled)
